(19) United States
(12) Patent Application Publication
(10) Pub. No.: US 2018/0044316 A1
(43) Pub. Date: Feb. 15, 2018

(54) POTENTIATORS OF BETA-LACTAM ANTIBIOTICS

(71) Applicant: UNIVERSITY OF NOTRE DAME DU LAC, South Bend, IN (US)

(72) Inventors: Shahriar MOBASHERY, Granger, IN (US); Marc A. BOUDREAU, Durham, NH (US)

(73) Assignee: UNIVERSITY OF NOTRE DAME DU LAC, South Bend, IN (US)

(21) Appl. No.: 15/555,851

(22) PCT Filed: Mar. 7, 2016
(86) PCT No.: PCT/US16/21251

§ 371 Date: Sep. 5, 2017

Related U.S. Application Data
(60) Provisional application No. 62/128,776, filed on Mar. 5, 2015.

(51) Int. Cl. C07D 401/04; A61K 45/06; C07D 401/14; C07D 413/14; A61K 31/454; C07D 403/04; A61K 31/506; C07D 233/64; A61K 31/4164; C07D 409/04; A61K 31/4178; C07D 277/22; A61K 31/426; C07D 417/04; C07D 405/14; A61K 31/4439

(52) U.S. Cl. CPC C07D 401/04; A61K 31/4439; A61K 45/06; C07D 401/14; C07D 413/14; A61K 31/454; C07D 403/04; A61K 31/506; C07D 233/64; A61K 31/4164; C07D 409/04; A61K 31/4178; C07D 277/22; A61K 31/426; C07D 417/04; C07D 405/14

(57) ABSTRACT

We disclose herein that the BlaR1 protein of methicillin-resistant Staphylococcus aureus (MRSA), an antibiotic sensor/signal transducer, is phosphorylated on exposure to β-lactam antibiotics. This event is critical for the onset of the biochemical events that unleash induction of antibiotic resistance. The BlaR1 phosphorylation and the antibiotic-resistance phenotype are abrogated in the presence of inhibitors described herein that restore susceptibility of the organism to β-lactam antibiotics. The invention thus provides compounds and methods for abrogating antibiotic resistance to β-lactam antibiotics and for treating infections causes by antibiotics prone to developing resistance.

### RELATED APPLICATIONS

This application claims priority under 35 U.S.C. § 119(e) to U.S. Provisional Patent Application No. 62/128,776, filed Mar. 5, 2015, which is incorporated herein by reference.

### GOVERNMENT SUPPORT

This invention was made with government support under Grant No. AI104987 awarded by the National Institutes of Health. The government has certain rights in the invention.

### BACKGROUND OF THE INVENTION

Staphylococcus aureus is a Gram-positive bacterium commonly found on the skin and in moist areas, such as the nasal cavity, yet it is often broadly resistant to many antibiotics. β-Lactam antibiotics were the drugs of choice for treatment of infection by S. aureus, but a variant of this organism, methicillin-resistant Staphylococcus aureus (MRSA) emerged in 1961, which exhibited resistance to the entire class of β-lactams. This organism has been a global clinical problem for over half a century. The molecular basis for the broad resistance of MRSA to β-lactams, which is incidentally inducible, was traced to a set of genes within the bla and mec operons. The BlaR1 (or the cognate MecR1) protein is a β-lactam antibiotic sensor/signal transducer, which communicates the presence of the antibiotic in the milieu to the cytoplasm in a process that is largely not understood (FIG. 1) (Staude et al., Biochemistry 2015, 54, 1600-1610). Signal transduction leads to activation of the cytoplasmic domain of BlaR1 (or MecR1), a zinc protease, which turns over the gene repressor BlaI (or MecI) in derepressing transcriptional events that result in expression of antibiotic-resistance determinants, the class A β-lactamase PC1 and/or the penicillin-binding protein 2a (PBP2a) (Llarrull and Mobashery, Biochemistry 2012, 51, 4642-4649).

An intriguing aspect of this system is its inducibility. Upon exposure to the antibiotic, the organism mobilizes. Once the antibiotic challenge is withdrawn, the system reverses itself. It was argued that when the signal for the presence of the antibiotic transduces to the cytoplasmic domain, the BlaR1 protein undergoes autoproteolysis, which unleashes the activity of the protease domain in degradation of the gene repressor BlaI. We have found that this autoproteolytic processing takes place in the absence of antibiotic as well. Therefore, proteolysis may lead to turnover of BlaR1 itself as an event in the reversal of induction. What is needed is the identification of what accounts for activation of the cytoplasmic domain toward degradation of BlaI in manifestation of the antibiotic-resistance response. Such identification could provide needed methods for reducing, preventing, or otherwise abrogating resistance to β-lactam antibiotics.

### SUMMARY

We disclose herein that the BlaR1 protein of methicillin-resistant Staphylococcus aureus (MRSA), an antibiotic sensor/signal transducer, is phosphorylated on exposure to β-lactam antibiotics. This event is critical for the onset of the biochemical events that unleash induction of antibiotic resistance. The BlaR1 phosphorylation and the antibiotic-resistance phenotype are abrogated in the presence of novel inhibitors that restore susceptibility of the organism to β-lactam antibiotics. The invention provides compounds, compositions, and methods for reducing, preventing, overcoming, and/or abrogating resistance to β-lactam antibiotics, and methods of treating bacterial infections caused by antibiotic resistant bacteria, particularly bacteria that can develop resistance to β-lactam antibiotics.

Accordingly, the invention provides compositions and methods for increasing the sensitivity of bacterial pathogens to antibiotics, including β-lactam antibiotics. In one embodiment, the invention provides a method for increasing the sensitivity of bacterial pathogens to β-lactam antibiotics by contacting the bacterial pathogen with one or more compounds described herein. In some embodiments the bacterial pathogen is MSRA. In other embodiments, the bacterial pathogen is Enterococcus faecalis.

The invention also provides compositions and methods for increasing the susceptibility of Gram positive or Gram negative pathogens to β-lactam antibiotics. Various embodiments provide pharmaceutical compositions, therapeutic formulations, product combination, or kits for use against MRSA infections comprising a compound described herein and one or more β-lactam antibiotics. The compounds and methods can be used for inhibiting the growth of bacteria, for example, Staphylococcus aureus. In some embodiments, the Staphylococcus aureus is resistant to, or sensitive to, methicillin, other β-lactams, macrolides, lincosamides, aminoglycosides, or a combination thereof. Thus, the invention further provides methods for increasing the sensitivity of Staphylococcus aureus to methicillin, other β-lactams, macrolides, lincosamides, or aminoglycosides. The methods can include administering an effective amount of a compound, a pair of compounds, or composition described herein.

The compounds described herein include a compound of Formula I:

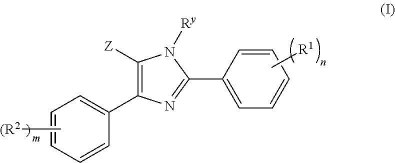

wherein

each R¹ is independently hydroxy, halo, (C₁-C₁₂)alkyl, (C₁-C₁₂)alkoxy, —CF₃, —OCF₃, —SH, —SMe, —N((C₂-C₈)alkyl)₂, or N-pyrrolidine;

each R² is independently H, hydroxy, halo, (C₁-C₁₂)alkyl, (C₁-C₁₂)alkoxy, —CF₃, or —OCF₃, or two R² groups form an oxadiazole;

R^{y} is H or (C₁-C₅)alkyl;

each n and m is independently 1, 2, 3, 4, or 5; and

Z is pyridyl, pyrimidinyl, thiophenyl, phenyl, or cyanophenyl;

or a pharmaceutically acceptable salt or solvate thereof.

In some embodiments, when Z is 4-pyridyl and R² is F in the para position, R¹ is not hydroxy, ethyl, isopropyl, tent-butyl, or —SMe in the para position. In various embodiments, when Z is 4-pyridyl and R² is F in the para position, R¹ is not H, hydroxy, methyl, ethyl, isopropyl, tert-butyl, —SMe, —SEt, —NMe₂, —S(O)Me, halo, —CF₃, phenyl, phenoxy, —OMe, —CO₂H, —NO₂, —NH₂, —NHSO₂Me, or —CN in the para position. In certain embodiments, Formula I and sub-formulas Formula II and Formula III below, exclude or can optionally exclude one or more of compounds 1, A2, A3, A6, A7, A8, A10, A11, A23, A24, A25, A27, A64, A65, A66, A67, A68, A70, A72, A73, A74, A76, A78, and/or A80, in any combination.

In some embodiments, Z is 4-pyridyl, 5-pyrimidinyl, 2-thiophenyl, 3-thiophenyl, phenyl, or 4-cyanophenyl.

Examples of compounds of Formula I include a compound of Formula II:

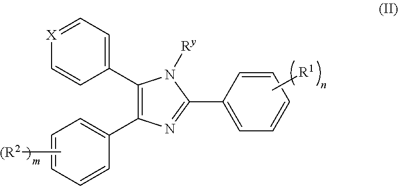

wherein

each R¹ is independently hydroxy, halo, (C₁-C₁₂)alkyl, (C₁-C₁₂)alkoxy, —CF₃, —OCF₃, —SH, —SMe, —N((C₂-C₈)alkyl)₂, or N-pyrrolidine;

each R² is independently H, hydroxy, halo, (C₁-C₁₂)alkyl, (C₁-C₁₂)alkoxy, —CF₃, or —OCF₃, or two R² groups form an oxadiazole;

R^{y} is H or (C₁-C₅)alkyl;

each n and m is independently 1, 2, 3, 4, or 5; and

X is N, CH, or C—CN;

or a pharmaceutically acceptable salt or solvate thereof.

One specific value of X is N. Other values for X include CH or C—CN.

One specific value of R^{y} is H. Another specific value of R^{y} is Me.

One specific value of R¹ is hydroxy. In other embodiments, R¹ can be (C₂-C₄)alkyl, such as ethyl, propyl, sec-propyl, iso-propyl, sec-butyl, or tert-butyl.

In some embodiments, R² is halo. One specific value of R² is fluoro.

In some embodiments, n is 1 or 2. In various embodiments, m is 1 or 2. In other embodiments, each n and m can be independently 2, 3, or 4. In certain specific embodiments, n is 1. In various specific embodiments, m is 1. In another embodiment, n is 1 and m is 1.

Examples of compounds of Formula II include a compound of Formula III:

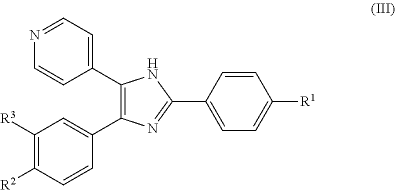

wherein

R¹ is hydroxy, halo, (C₁-C₁₂)alkyl, (C₁-C₁₂)alkoxy, —CF₃, —OCF₃, —SH, —SMe, —N((C₂-C₈)alkyl)₂, or N-pyrrolidine;

R² is H, hydroxy, halo, (C₁-C₁₂)alkyl, (C₁-C₁₂)alkoxy, —CF₃, or —OCF₃; and

R³ is H or F; or

R² and R³ together form an oxadiazole;

or a pharmaceutically acceptable salt or solvate thereof.

The invention also provides a composition comprising a compound described herein, in combination with a β-lactam antibiotic. In one embodiment, the β-lactam antibiotic is oxacillin. In another embodiment, the β-lactam antibiotic is ceftaroline.

The invention further provides a method to reverse the methicillin-resistant phenotype in BlaR1 comprising contacting methicillin-resistant Staphylococcus aureus (MRSA) with an effective amount of a compound described herein, thereby rendering MRSA susceptible to β-lactam antibiotics.

The invention yet further provides a method to inhibit or kill methicillin-resistant Staphylococcus aureus (MRSA) comprising contacting the MRSA with an amount of a compound described herein effective to reverse the methicillin-resistant phenotype in BlaR1, and contacting the MRSA with an effective antibacterial amount of a β-lactam antibiotic.

In another embodiment, the invention provides a method to lower the degree of phosphorylation of BlaR1 comprising contacting a bacteria having BlaR1 with an effective amount of a compound described herein.

In yet another embodiment, the invention provides a method to attenuate the minimum inhibitory concentration (MIC) of a β-lactam antibiotic comprising contacting a bacterium with an effective amount of a compound described herein in combination with contacting the bacterium with a β-lactam antibiotic. The invention therefore provides for the use of a compound described herein for preparing a medicament to treat a bacterial infection. The bacterial infection can be, for example, a methicillin-resistant Staphylococcus aureus (MRSA) infection.

Further embodiments relate to methods of ameliorating and/or treating a bacterial infection that can include administering to a subject suffering from the bacterial infection an effective amount of one or more compounds of Formulas I-III, or a pharmaceutical composition that includes one or more compounds of Formulas I-III, or a pharmaceutically acceptable salt thereof. Other embodiments described herein relate to using one or more compounds of Formulas I-III in the manufacture of a medicament for ameliorating and/or treating a bacterial infection. Still other embodiments described herein relate to compounds of Formulas I-III that can be used for ameliorating and/or treating a bacterial infection. Other embodiments relate to methods of ameliorating and/or treating a bacterial infection that can include administering to a patient infected with the bacterial infection an effective amount of one or more compounds of Formulas I-III. Some embodiments described herein relate to methods of inhibiting the replication of a bacteria that can include administering to a patient infected with the bacteria an effective amount of one or more compounds of Formulas I-III. In one embodiment, the bacterial infection can be an S. aureus infection, for example, a MRSA infection.

The invention thus provides novel compounds of Formulas I-III, intermediates for the synthesis of compounds of Formulas I-III, as well as methods of preparing compounds of Formulas I-III. The invention also provides compounds of Formulas I-III that are useful as intermediates for the synthesis of other useful compounds. The invention provides for the use of the compounds and compositions described herein in medical therapy. The compounds of Formulas I-III can be used in the manufacture of medicaments useful for the treatment of bacterial infections in a mammal, such as a human. Compositions and medicaments described herein can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. The bla system includes the β-lactam-antibiotic sensor/signal transducer protein BlaR1, which is acylated by β-lactam antibiotics in the extracellular sensor domain (BlaR^{s}). This initiates signal transduction through the membrane to the proteolytic domain (cytBlaR), which autoproteolyzes at S283-F284. The BlaI repressor protein binds to the bla operon, which is comprised of genes that encode BlaI, BlaR1, and the PC1 β-lactamase (blaZ). Degradation of BlaI by the cytoplasmic protease domain of BlaR1 leads to derepression and transcription of the genes. BlaR1 is phosphorylated on the cytoplasmic side, at least at two amino acids, in response to the exposure of S. aureus to β-lactam antibiotics. The cognate mec operon encodes the corresponding MecI (gene repressor), MecR1 (antibiotic sensor/signal transducer) and MecA (for penicillin-binding protein 2a, the antibiotic resistance determinant).

FIG. 2. Western-blot analysis of NRS128 whole-cell extract grown in the absence and presence of 10 μg/mL CBAP using antibodies against phosphothreonine, phosphotyrosine, and phosphoserine. The ˜30 kDa bands seen with anti-phosphotyrosine and anti-phosphoserine correspond to fragmented BlaR1. No such band was detected using anti-phosphothreonine antibody. The bands between 36.5 kDa and 97.4 kDa are attributed to the ubiquitous Protein A, which was cleared from the extract in subsequent experiments.

FIGS. 3A-C. Western-blot analysis of CBAP-induced (+) and non-induced (−) extracts of NRS128 after immunoprecipitation with anti-BlaR^{s}-agarose. Nitrocellulose membrane containing unbound (“UB”) and eluted bound (“B”) fractions were probed with (A) anti-BlaR^{s} or (B) anti-P-Tyr. The arrow in (A) indicates the C-terminal fragment of BlaR1, seen only in the bound fraction. The arrows in (B) and (C) indicate the unbound N-terminal fragment containing the phosphorylated cytoplasmic domain. Bands identified by anti-BlaR^{s} between 40 kDa and 50 kDa are undefined proteolytic fragments of BlaR1 (see Dzhekieva et al., Biochemistry 2012, 51, 2804-2811). (C) Western-blot analysis of CBAP-induced whole-cell extract using antibody against phosphoserine (anti-P-Ser) is shown. Both the full-length and N-terminal fragment of BlaR1 (arrow) were detected.

FIGS. 4A-B. BlaR1 phosphorylation in the absence and presence of 7 or 17 μg/mL of compound 1. Whole-cell extracts of NRS128 were cleared of Protein A by incubation with IgG Sepharose and analyzed by Western blot using antibodies against (A) phosphotyrosine and (B) phosphoserine.

FIGS. 5A-B. The effect of compounds 10, 11, or 12 on BlaR1 tyrosine phosphorylation and β-lactamase activity. (A) Whole-cell extracts of NRS70 grown in the absence or presence of 0.7 or 7 μg/mL compounds 10, 11, or 12 were cleared of Protein A by incubation with IgG Sepharose and analyzed by western blot using antibodies against phosphotyrosine. (B) β-Lactamase activity of the culture media after induction with CBAP in the absence or presence of 7 μg/mL compounds 10, 11, or 12 was measured by monitoring hydrolysis of nitrocefin at A₅₀₀ and normalized to the activity in the absence of inhibitor.

FIG. 6. The effect of compounds 10, 11, or 12 (0, 7 or 17 μg/mL) on serine phosphorylation of BlaR1 fragment. NRS70 whole-cell extracts were cleared of Protein A and analyzed by Western Blot using antibody against phosphoserine.

FIG. 7. SDS-PAGE of purified Stk1 from S. aureus strain NRS70.

FIGS. 8A-F. Inhibition of autophosphorylation of purified Stk1 or myelin basic protein (MBP) by compounds 10-12. Purified Stk1 or myelin basic protein (MBP) was radiolabelled by [γ-³²P]-ATP (20 μM) in the presence of increasing concentrations of synthetic inhibitors 10-12. Inhibition of Stk1 autophosphorylation by 10-12, giving IC₅₀ values of 3.1±0.8 μg/mL (8.8±2.4 μM), 5.1±1.4 μg/mL (14.7±4μM), and 6.3±1.3 μg/mL (18±3.8 μM), respectively (panels A, C, and E). Inhibition of MBP phosphorylation by compounds 10-12, giving IC₅₀ values of 2.1±0.6 μg/mL (6.1±1.8 μM), 4.2±1.3 μg/mL (12±3.6 μM), and 5.7±1.3 μg/mL (16.2±3.7 μM), respectively (panels B, D, and F).

### DETAILED DESCRIPTION

The BlaR1 protein of methicillin-resistant Staphylococcus aureus (MRSA), an antibiotic sensor/signal transducer, is phosphorylated on exposure to β-lactam antibiotics. This event is critical for the onset of the biochemical events that unleash induction of antibiotic resistance. The BlaR1 phosphorylation and the antibiotic-resistance phenotype are abrogated in the presence of novel inhibitors described herein, which inhibitors restore susceptibility of the organism to β-lactam antibiotics. The invention thus provides compounds and methods for abrogating antibiotic resistance to β-lactam antibiotics.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the end-points of a recited range as discussed above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “alkyl” refers to a straight- or branched-chain alkyl group having from 1 to about 20 carbon atoms in the chain. For example, the alkyl group can be a (C₁-C₂₀)alkyl, a (C₁-C₂)alkyl, (C₁-C₈)alkyl, (C₁-C₆)alkyl, or (C₁-C₄)alkyl. Examples of alkyl groups include methyl (Me), ethyl (Et), n-propyl, isopropyl, butyl, isobutyl, sec-butyl, tert-butyl (t-Bu), pentyl, isopentyl, tent-pentyl, hexyl, isohexyl, and groups that in light of the ordinary skill in the art and the teachings provided herein would be considered equivalent to any one of the foregoing examples. Alkyl groups can be optionally substituted or unsubstituted, and optionally partially unsaturated, such as in an alkenyl group.

The term “halogen” refers to chlorine, fluorine, bromine or iodine. The term “halo” refers to chloro, fluoro, bromo or iodo.

As to any of the groups or “substituents” described herein (e.g., groups R¹ and R²), each can further include one or more (e.g., 1, 2, 3, 4, 5, or 6) substituents. It is understood, of course, that such groups do not contain any substitution or substitution patterns which are sterically impractical and/or synthetically non-feasible.

The term “substituted” means that a specified group or moiety can bear one or more (e.g., 1, 2, 3, 4, 5, or 6) substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted in some embodiments but can be substituted in other embodiments. In other words, the variables R¹, R², and R³ and their elements can be optionally substituted. In various embodiments, suitable substituent groups (e.g., on groups R¹, R², and R³ and/or their elements) include one or more of alkyl, alkenyl, alkynyl, alkoxy, halo, haloalkyl, hydroxy, hydroxyalkyl, aryl, aroyl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl (alkyl)amine, and/or cyano. In certain embodiments, any one of the above groups can be included or excluded from a variable (e.g., groups R¹ and R²) or from a group of substituents.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo (e.g., by administration to a patient).

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

For example, the term “effective amount” can refer to an amount of compound or composition, which upon administration, is capable of reducing or preventing proliferation of a bacteria, reducing or preventing symptoms associated with a bacterial infection, reducing the likelihood of bacterial infection, or preventing bacterial infection. Typically, the subject is treated with an amount of a therapeutic composition sufficient to reduce a symptom of a disease or disorder, such as an infection, by at least about 25%, about 50%, about 75%, or about 90%.

The terms “treating”, “treat” and “treatment” can include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

Treatments may be reactive, such as for combating an existing infection, or prophylactic, for preventing infection in an organism susceptible to infection. In some embodiments, compositions can be used to treat infections by drug-resistant strains of bacteria, for example MRSA (methicillin resistant S. aureus), MRSE (methicillin resistant S. epidermidis), PRSP (penicillin resistant S. pneumoniae), VIRSA (vancomycin intermittently resistant Staphylococcus aureus) or VRE (vancomycin resistant Enterococci). The term “drug-resistant” a condition where the bacteria are resistant to treatment with one or more conventional antibiotics, particularly β-lactam antibiotics. Accordingly, the invention provides a method for killing or inhibiting growth of gram positive bacteria comprising contacting gram positive bacteria with a compound or composition described herein, thereby killing or inhibiting the growth of the bacteria. The contacting can be performed in vivo in a human or animal, or in vitro, for example, in an assay. The gram positive bacteria can be of the genus Enterococcus or Staphylococcus. In certain embodiments, the bacteria is a drug-resistant strain of the genus Staphylococcus. In certain specific embodiments, the bacteria is a methicillin-resistant Staphylococcus aureus (MRSA) strain.

In some embodiments, the bacterial infection may be due to Gram-positive bacteria, including, but not limited to, methicillin resistant Staphylococcus aureus (MRSA), community-acquired methicillin resistant Staphylococcus aureus (CAMRSA), vancomycin-intermediate-susceptible Staphylococcus aureus (VISA), methicillin-resistant coagulase-negative staphylococci (MR-CoNS), vancomycin-intermediate-susceptible coagulase-negative staphylococci (VI-CoNS), methicillin susceptible Staphylococcus aureus (MSSA), Streptococcus pneumoniae (including penicillin-resistant strains [PRSP]) and multi-drug resistant strains [MDRSP]), Streptococcus agalactiae, Streptococcus pyogenes and Enterococcus faecalis. In particular embodiments, the bacterial infection may include, but is not limited to, complicated skin and skin structure infections (cSSSI); community acquired pneumonia (CAP); complicated intra-abdominal infections, such as, complicated appendicitis, peritonitis, complicated cholecystitis and complicated diverticulitis; uncomplicated and complicated urinary tract infections, such as, pyelonephritis; and respiratory and other nosocomial infections.

The term “infection” refers to the invasion of the host by germs (e.g., bacteria) that reproduce and multiply, causing disease by local cell injury, release of poisons, or germ-antibody reaction in the cells. The compounds and compositions described herein can be used to treat a gram positive bacterial infection, for example, an infection in a mammal, such as a human.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

Phosphorylation of BlaR1 in Manifestation of the Methicillin-Resistance Phenotype in Staphylococcus aureus and its Abrogation by Small Molecules

We have now discovered that BlaR1 experiences phosphorylation at a minimum of one serine and one tyrosine in the cytoplasmic domain on exposure to β-lactam antibiotics. We also document that inhibition of this phosphorylation by small molecules reverses the methicillin-resistant phenotype, rendering MRSA susceptible to β-lactam antibiotics. Thus, the BlaR1 phosphorylation is an important step in regulation of the function of the protein, critical for the manifestation of the antibiotic-resistance phenotype. Whereas protein phosphorylation and its contribution to many regulatory events are widely known in eukaryotes, the same information relating to bacteria is significantly less understood. Nonetheless, S. aureus appears to have at least five protein kinases, which would contribute to these processes.

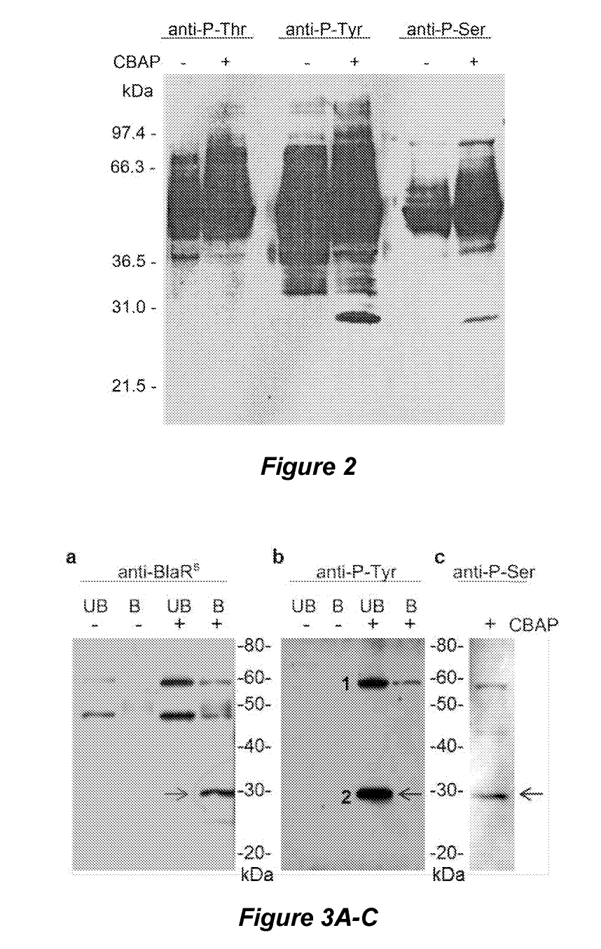

We investigated phosphorylation of BlaR1 in strain Staphylococcus aureus NRS128 (also designated as NCTC8325) on exposure to β-lactam antibiotics. This strain, which has the bla but not the mec operon, was grown in the absence or in the presence of 10 μg/mL CBAP (2-(2′-carboxyphenyl)-benzoyl-6-aminopenicillanate), a good penicillin inducer of resistance (Llarrull et al., J Biol. Chem., 2011, 286, 38148-38158). In a series of experiments that are outlined in the Examples below, we document by Western-blot analysis using anti-phosphotyrosine and anti-phosphoserine antibodies that the cytoplasmic domain of BlaR1 is phosphorylated at least on one tyrosine and one serine residue (FIGS. 2 and 3). The same experiment performed with an anti-phosphothreonine antibody documented the absence of threonine phosphorylation.

If phosphorylation of BlaR1 is important for the manifestation of resistance, it would be significantly valuable for treating bacterial infections if resistance to β-lactam antibiotics could be attenuated (or reversed) in the presence of protein-kinase inhibitors. The strain NRS128, used above, is not a PBP2a-dependent strain, hence we substitute it with S. aureus MRSA252 (also known as USA200) for the following experiments. This strain exhibits high-level resistance to β-lactam antibiotics due to its expression of PBP2a. Its genome has been sequenced (Holden et al., Proc. Natl. Acad. Sci. USA., 2004, 101, 9786-9791) and it harbors the transposon Tn552, which encodes BlaR1. BlaR1 of MRSA252 has 99% sequence identity to that of NRS128. The minimal-inhibitory concentration (MIC) of oxacillin (a penicillin) against this strain is 256 μg/mL, consistent with high-level resistance.

A protein-kinase inhibitor library of 80 known compounds was tested in a 96-well format against strain MRSA252 for the screening. We first determined MICs (broth microdilution method) (CLSI, Performance Standards for Antimicrobial Susceptibility Testing; Twenty-Second Informational Supplement. CLSI document M100-S22. Clinical and Laboratory Standards Institute; Wayne, Pa.) for all the inhibitors in the library, in case some of them might have antibacterial properties of their own, which could complicate the analysis.

Indeed, a few of these compounds did exhibit modest antibacterial activity on their own, and were not studied further. Subsequently, we investigated the bacterial growth in the presence of oxacillin concentrations of 256 (MIC), 128 (½ MIC), and 64 (¼ MIC) μg/mL and one of two fixed concentrations of the protein-kinase inhibitors without antibiotic property (0.7 μg/mL or 7 μg/mL). This rapid initial screening identified compound 1 as meeting the selection criteria of lowering the MIC for oxacillin, which was followed up by the actual evaluation of the MIC of oxacillin against S. aureus MRSA252 in the presence of the inhibitor. Inhibitor 1 at 7 μg/mL produced a reproducible four-fold decrease in the MIC of oxacillin for S. aureus MRSA252. The MIC of the kinase inhibitor alone against the same organism was ≧64 μg/mL.

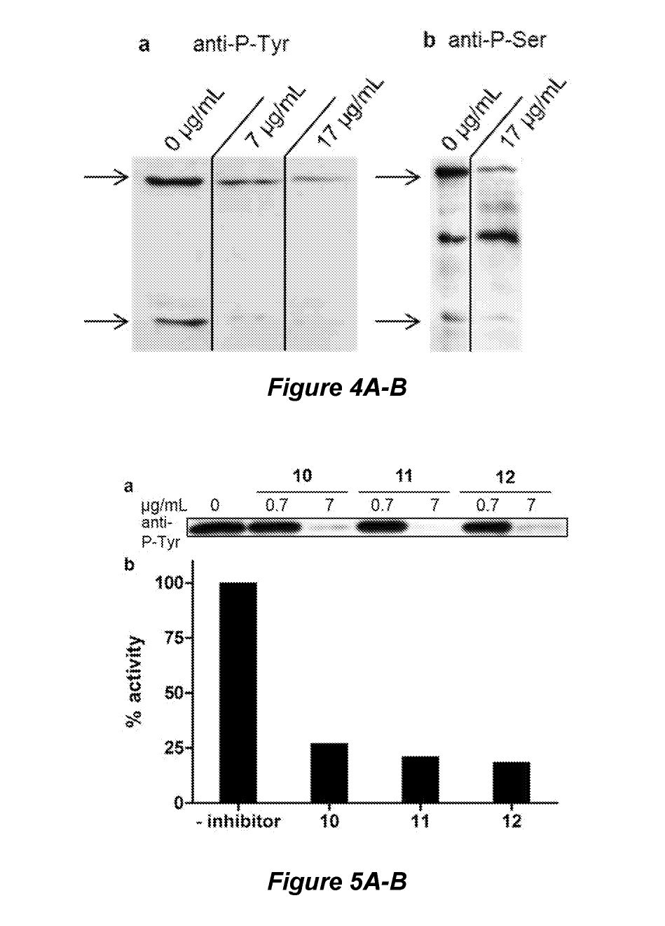

Next, we tested the effect of exposure of CBAP-induced NRS128 to this kinase inhibitor. Cultures were induced with 10 μg/mL CBAP in the presence of 0, 7, or 17 μg/mL of compound 1. Whole-cell extracts of these bacteria were analyzed for BlaR1 phosphorylation by western blot using anti-Phos-Ser and anti-Phos-Tyr antibodies. Compound 1 inhibited both the phosphotyrosine and phosphoserine kinase activities by as much as 70-90% (FIG. 4). Hence, compound 1 lowered the degree of phosphorylation of BlaR1, and at the same time, the MIC for oxacillin was attenuated.

Compound 1 is a known mammalian serine/threonine-kinase inhibitor (Frantz et al., Biochemistry, 1998, 37, 13846-13853). That this compound inhibited the formation of phosphoserine and phosphotyrosine moieties in the BlaR1 protein was an important observation indicating that the kinase domain had a distinct structure that might make it a useful target for drug discovery. We undertook optimization of the structure of compound 1 for inhibition of the bacterial protein kinase(s) that phosphorylates BlaR1. More than 75 structural variants of compound 1 were synthesized and screened for the ability to lower the MIC for oxacillin and other β-lactam antibiotics. This involved diversification of the imidazole core at substituents attached to positions 2, 4, and 5.

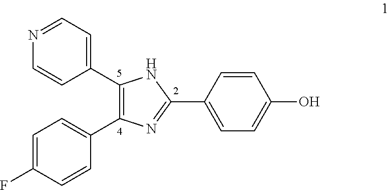 [SMILES: Oc1ccc(-c2nc(-c3ccc(F)cc3)c(-c3ccncc3)[nH]2)cc1]

Diversification at the imidazole C2 position was achieved using the methodology of Gallagher et al. for construction of imidazoles (Bioorg. Med. Chem. 1997, 5, 49-64), as shown in Scheme 1. Briefly, compound 2 was treated with LDA, followed by Weinreb amide 3, to give the ketone 4. This intermediate was then allowed to react with various benzaldehydes in the presence of copper(II) acetate and ammonium acetate to give a library of C₂-modified imidazoles.

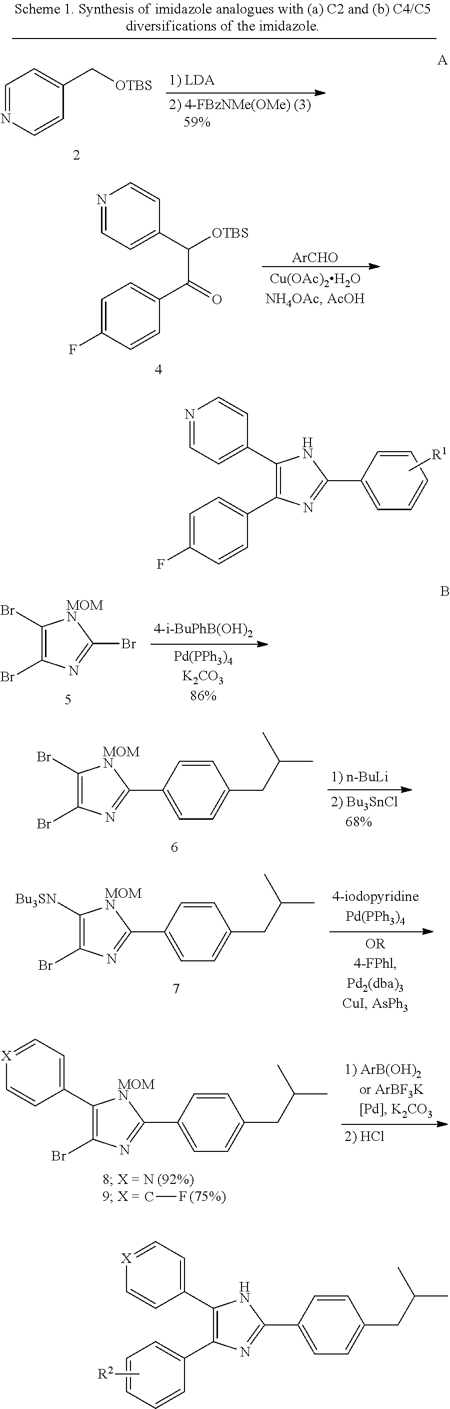

For diversification at C4 and C5, we used metal-catalyzed coupling to sequentially install the desired rings onto the imidazole (Scheme 1). Thus, tribromoimidazole derivative 5 (Niculescu-Duvaz et al., Bioorg. Med. Chem. 2010, 18, 6934-6952) was subjected to a Suzuki reaction with 4-isobutylphenylboronic acid to give C₂-substituted imidazole 6. Suzuki reactions performed on 4,5-dibromoimidazoles such as 6 usually result in a mixture of mono- and di-coupled products (Recnik et al., Synthesis 2013, 45, 1387-1405). Therefore, 6 was converted to stannane 7 by lithiation and quenching with tributyltin chloride. Stannane 7 smoothly underwent Stille coupling (Liverton et al., J. Med. Chem. 1999, 42, 2180-2190) with either 4-iodopyridine or 4-fluoroiodobenzene to give 8 and 9, respectively. These intermediates were then subjected to Suzuki reactions with arylboronic acids or potassium aryltrifluoroborate salts, followed by deprotection to give the desired imidazole analogues.

We evaluated the new imidazole analogues for their ability to lower the MIC of oxacillin against MRSA in the same manner as for the kinase inhibitor library. In addition to MRSA252, we expanded our investigation to include strains NRS123 and NRS70, both of which have 95% sequence identities between their BlaR1 proteins and that of NRS128, with the bla operon encoded on plasmids (pMW2 and pN315, respectively). Interestingly, NRS123 also encodes for a truncated, nonfunctional MecR1 protein and lacks the gene for MecI, therefore PBP2a expression in this strain is regulated by the bla operon. The MIC of oxacillin against the resistant MRSA strains NRS123 and NRS70 are 16 and 32 μg/mL, respectively. Our inhibitors 10-12 exhibited remarkably improved activity in lowering the MIC of oxacillin. Notably, compounds 10-12 at 7 μg/mL are active across all three MRSA strains (Table 1).

TABLE 1
MIC values of oxacillin (μg/mL) against MRSA strains in the absence
of a kinase inhibitor and in the presence of compounds 10-12 at 7 μg/mL.

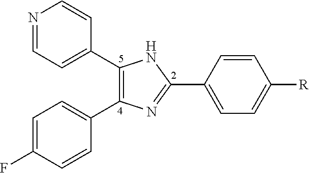

| Strain | No inhibitor | 10 | 11 | 12 |
|---|---|---|---|---|
| MRSA252 | 256 | 2 | 16 | 4 |
| NRS123 | 16 | 8 | 4 | 4 |
| NRS70 | 32 | 4 | 0.5 | 0.5 |

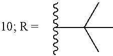 [SMILES: *C(C)(C)C]

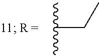 [SMILES: *CC]

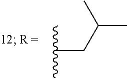 [SMILES: *CC(C)C]

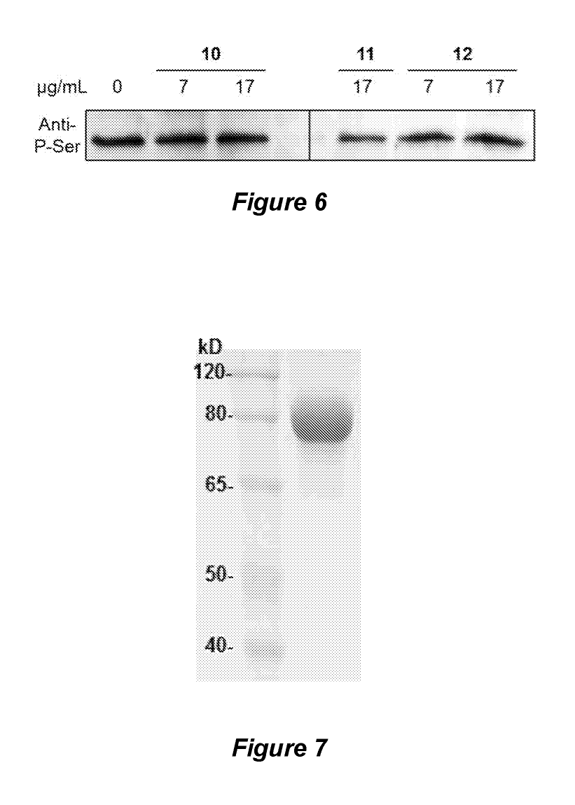

We then evaluated the ability of compounds 10, 11 or 12 to inhibit tyrosine phosphorylation of BlaR1 in NRS70 extracts. The bacteria were grown under the conditions described earlier for NRS128, induced by CBAP in the absence and presence of 0.7 μg/mL or 7 μg/mL compounds 10, 11 or 12, and analyzed by western blot using antibody against phosphotyrosine (FIG. 5a). The presence of 7 μg/mL inhibitor almost completely abolished tyrosine phosphorylation of the BlaR1 fragment in all cases. However, serine phosphorylation was not inhibited by any of these compounds, even at 17 μg/mL (FIG. 6), in contrast to the case of 1, which had inhibited both. This provides evidence for the critical nature of tyrosine phosphorylation for regulation of BlaR1.

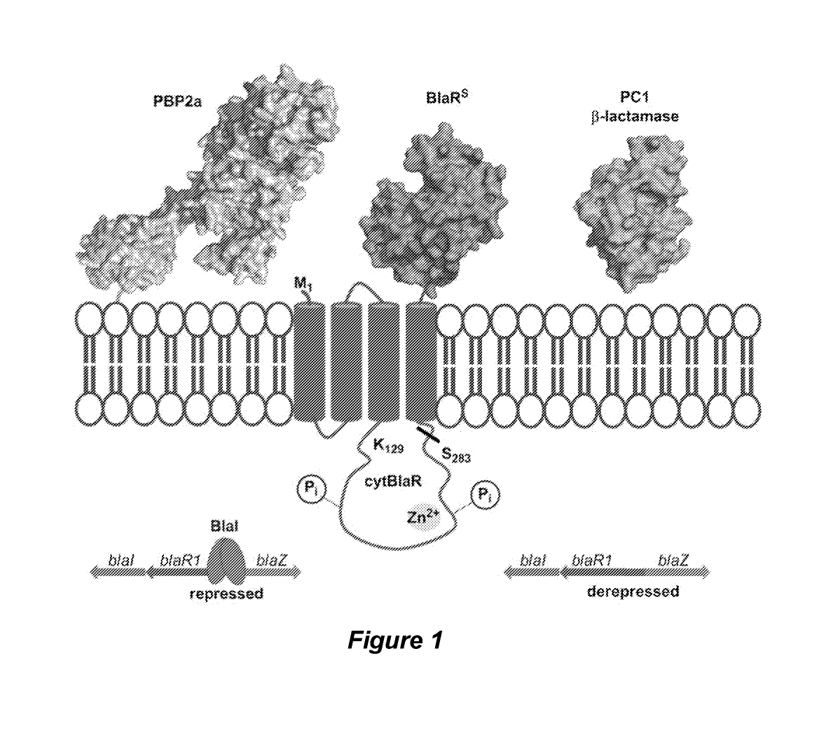

As we propose that phosphorylation is an activation step for the bla system, abrogation of phosphorylation should have an effect on expression of the resistance determinant(s). We have shown this to be the case in attenuation of the level of β-lactamase (product of the blaZ gene, FIG. 1). To document this, we monitored hydrolysis of the chromogenic β-lactam nitrocefin at A₅₀₀ by β-lactamase expressed in the NRS70 culture media, according to the methodology reported previously (Llarrull et al., J. Biol. Chem. 2011, 286, 38148-38158). The initial rates of the reaction in the absence and presence of 7 μg/mL compound 10, 11 or 12 were normalized to the activity in the absence of inhibitor (FIG. 5b). As expected, the presence of compounds 10, 11 or 12 decreased the β-lactamase activity by ˜70-80%, congruent with the MIC data and western-blot analysis.

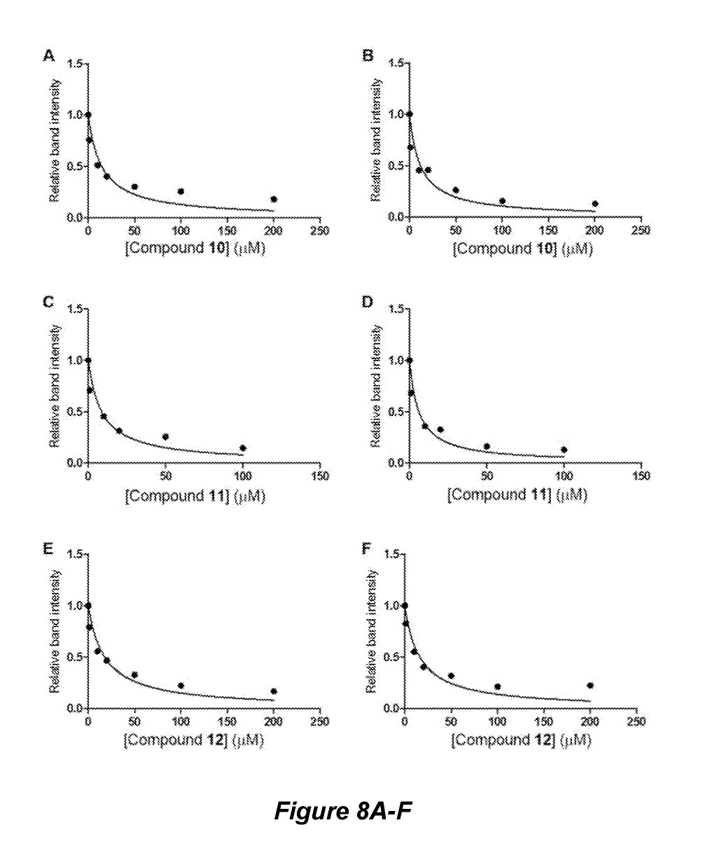

An observation by Tamber et al. that an stk1(pknB) gene knockout strain of the USA300 strain showed lower MIC values for β-lactam antibiotics is of interest (Tamber et al., Infect. Immun. 2010, 78, 3637-46). The gene pknB (also known as stk1) encodes a highly-conserved broad-specificity protein kinase in S. aureus that phosphorylates its substrates on serine, threonine or tyrosine. To confirm Stk1 is a protein target of the inhibitors in this study, we cloned the gene and expressed and purified Stk1 from S. aureus NRS70 (FIG. 7). We found that compounds 10, 11, and 12 inhibit Stk1 autophosphorylation with IC₅₀ values of 3.1±0.8 μg/mL, 5±1 μg/mL, and 6±1 μg/mL, respectively. The compounds also inhibit phosphorylation of myelin-basic protein (MBP) by Stk1, with IC₅₀ values of 2.1±0.6 μg/mL, 4±1 μg/mL, and 6±1 μg/mL, respectively (FIG. 8).

In conclusion, we have shown that the BlaR1 protein of MRSA is phosphorylated at a minimum of one serine and one tyrosine in response to challenge by β-lactam antibiotics, a step that is crucial in the signaling events leading to the induction of antibiotic resistance. The breadth of kinase/phosphatase-regulated processes in bacteria is likely vastly greater than is appreciated presently. This BlaR1 study represents the first insight as to the molecular-level regulation of a key resistance pathway in an important human pathogen. The documentation that inhibition of phosphorylation by small molecules reverses the MRSA phenotype makes available a new strategy to bring β-lactam antibiotics back from obsolescence in treatment of this insidious organism (Scheme 2, below).

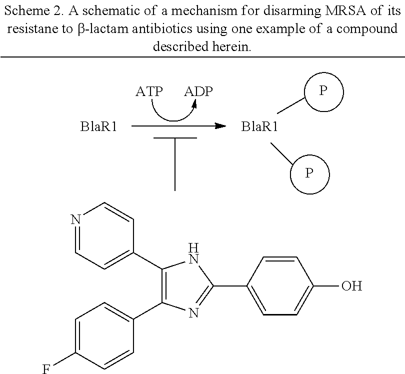

### Combination Therapy

The compounds described herein may be administered alone or in combination with other therapeutic agents, such as antibiotic, anti-inflammatory or antiseptic agents such as anti-bacterial agents, anti-fungicides, anti-viral agents, and anti-parasitic agents. In some embodiments, a pharmaceutical composition comprises one or more compounds described herein and one or more antibiotic or antiseptic agents. Examples of suitable active agents include penicillins, cephalosporins, carbacephems, cephamycins, carbapenems, monobactams, aminoglycosides, glycopeptides, quinolones, tetracyclines, macrolides, and fluoroquinolones. Suitable antiseptic agents that can be used include iodine, silver, copper, chlorhexidine, polyhexanide and other biguanides, chitosan, acetic acid, and hydrogen peroxide. These agents may be incorporated as part of the same pharmaceutical composition or may be administered separately (concurrently or sequentially). The pharmaceutical compositions may also contain anti-inflammatory drugs such as steroids and macrolactam derivatives.

Several embodiments described herein relate to a pharmaceutical composition that includes one or more β-lactam antibiotics and one or more compounds described herein. β-Lactam antibiotics are bactericidal, and can act by inhibiting the synthesis of the peptidoglycan layer of bacterial cell walls. The peptidoglycan layer is important for cell wall structural integrity, especially in Gram-positive bacteria. Examples of β-lactam antibiotics include, but are not limited to, benzathine penicillin, benzylpenicillin (penicillin G), phenoxymethylpenicillin (penicillin V), procaine penicillin, methicillin, oxacillin, nafcillin, cloxacillin, dicloxacillin, flucloxacillin, temocillin, amoxicillin, ampicillin, co-amoxiclav, azlocillin, carbenicillin, ticarcillin, mezlocillin, piperacillin, cephalosporins, cephalexin, cephalothin, cefazolin, cefaclor, cefuroxime, cefamandole, cephamycins, cefotetan, cefoxitin, ceftriaxone, cefotaxime, cefpodoxime, cefixime, ceftazidime, cefepime, cefpirome, imipenem, meropenem, ertapenem, faropenem, doripenem, monobactams, aztreonam, tigemonam, nocardicin A, and tabtoxinine-β-lactam.

Some embodiments provide methods for inhibiting the growth and/or reproduction of susceptible organisms, and/or to increasing the sensitivity of susceptible organisms to β-lactam antibiotics. Susceptible organisms generally include gram positive and gram negative, aerobic and anaerobic organisms whose growth can be inhibited by embodiments described herein. Susceptible organisms include, but are not limited to, Staphylococcus, Lactobacillus, Streptococcus, Streptococcus agalactiae, Sarcina, S. pneumoniae, S. pyogenes, S. mutans, Escherichia, Enterobacter, Klebsiella, Pseudomonas, Pseudomonas aeruginosa, Acinetobacter, Proteus, Campylobacter, Citrobacter, Nisseria, Bacillus anthraces, Bacillus cereus, Bacillus subtilis, Bacteroides, Peptococcus, Clostridium, Salmonella, Shigella, Serratia, Haemophilus, Brucella, Mycobacterium tuberculosis and similar organisms.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. Nos. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The invention provides therapeutic methods of treating a bacterial infection in a mammal, which involve administering to a mammal having a bacterial infection an effective amount of a compound or composition described herein. A mammal includes a primate, human, rodent, canine, feline, bovine, ovine, equine, swine, caprine, bovine and the like. The ability of a compound of the invention to treat a bacterial infection may be determined by using assays well known to the art.

The invention also provides a kit comprising a packaging containing one or more doses of a first pharmaceutical formulation comprising a compound described herein or a pharmaceutically acceptable salt thereof, and one or more doses of a second pharmaceutical formulation comprising an antibiotic, each together with written instructions directing the co-administration of the first pharmaceutical formulation and the second pharmaceutical formulation for the treatment of bacterial infection. In some embodiments, the first dose of the first pharmaceutical formulation comprises a loading dose of a compound described herein. In some embodiments, the first dose of the second pharmaceutical formulation comprises a loading dose of an antibiotic. The individual doses of the pharmaceutical formulations, can independently be in any dosage form, e.g. tablets, capsules, solutions, creams, etc. and packaged within any of the standard types of pharmaceutical packaging materials, e.g. bottles, blister-packs, IV bags, syringes, etc., that may themselves be contained within an outer packaging material such as a paper/cardboard box. In some embodiments, the kit further comprises one or more of culture media, culture plates, PCR primers, test strips, and stains for identifying the infective agent.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Preparation of Phosphorylation Inhibitors

General information. Reagents for chemical synthesis were purchased from Sigma-Aldrich Chemical Co. (St. Louis, Mo., U.S.A.) or Alfa Aesar (Ward Hill, Mass., U.S.A.). ¹H and ¹³C NMR spectra were acquired on a Varian DirectDrive 600 or a Varian INOVA-500 NMR spectrometer. High-resolution mass spectra were acquired on a Bruker microTOF/Q2 mass spectrometer (Bruker Daltonik, Bremen, Germany) by electrospray ionization. Thin-layer chromatography was done on EMD Millipore (Billerica, Mass., U.S.A.) 0.25 mm silica gel 60 F₂₅₄ plates. Column chromatography was done either manually using silica gel 60, 230-400 mesh (40-63 μm particle size) purchased from Sigma-Aldrich Chemical Co., or on a Teledyne Combiflash Rf 200i automated chromatography system (Teledyne Isco, Lincoln, Nebr., U.S.A.) using disposable silica gel columns. The known compounds, 4-(((tert-butyldimethylsilyl)oxy)methyl)pyridine (5), 4-fluoro-N-methoxy-N-methylbenzamide (6), 2-(((tert-butyldimethylsilyl)oxy)-1-(4-fluorophenyl)-2-(pyridin-4-yl)ethan-1-one (7), and 1-methoxymethyl-2,4,5-tribromoimidazole (8) were synthesized according to literature procedures (see for example, Gallagher, T. F. et al. Bioorg. Med. Chem. 5, 49-64 (1997) and Niculescu-Duvaz, D. et al. Bioorg. Med. Chem. 18, 6934-6952 (2010)).

General procedure for synthesis of triarylimidazole analogues with variation at C2 of the imidazole ring (Method A). A literature procedure was followed (Liverton, N.J. et al. J. Med. Chem. 42, 2180-2190 (1999)). Compound 4 (1.0 equiv.), the aldehyde (1.1 equiv.), Cu(OAc)₂.H₂O (0.3 equiv.), and NH₄OAc (10 equiv.) were dissolved in AcOH (7.5 mL/mmol 7), and the mixture was stirred at 110° C. for 1.5 h. The solution was then cooled to room temperature and was added to a mixture of conc. NH₄OH (3×volume of AcOH used) and ice. After stirring for 10 minutes, the mixture was extracted with EtOAc, and the combined organic layer was washed with brine. The organic solution was dried over anhydrous Na₂SO₄, the suspension was filtered, and the solvent of the filtrate was removed in vacuo. Purification of the residue by flash chromatography gave the desired products in typical yields of 40-50%.

2-(4-tert-Butylphenyl)-4-(4-fluorophenyl)-5-(4-pyridyl)imidazole (10). This product was synthesized by reacting 4 with 4-tert-butylbenzaldehyde according to Method A. Purification by flash chromatography (silica, 100% CH₂Cl₂ to 95:5 CH₂Cl₂/MeOH) gave the product as a yellow powder (49%). ¹H NMR (600 MHz, CD₃OD) δ1.37 (s, 9H, C(CH₃)₃), 7.20 (t, 2H, J=8.8 Hz, ArH), 7.52-7.56 (m, 6H, ArH), 7.93 (d, 2H, J=8.8 Hz, ArH), 8.43 (br s, 2H, ArH); ¹³C NMR (150 MHz, CD₃OD) δ31.8, 35.8, 117.0 (²J_{CF}=21.3 Hz), 123.3, 125.7, 127.0, 127.1, 128.2, (⁴J_{CF}=2.2 Hz), 129.3, 130.3, 132.2 (³J_{CF}=7.9 Hz), 143.5, 149.6, 150.3, 153.9, 164.4 (¹J_{CF}=246.8 Hz); HRMS (ESI): calcd for C₂₄H₂₃FN₃ 372.1871, found 372.1881 [MH]⁺.

2-(4-Ethylphenyl)-4-(4-fluorophenyl)-5-(4-pyridyl)imidazole (11). This product was synthesized by reacting 4 with 4-ethylbenzaldehyde according to Method A. Purification by flash chromatography (silica, 100% CH₂C₁₂ to 95:5 CH₂Cl₂/MeOH) gave the product as a yellow powder (48%). ¹H NMR (600 MHz, CD₃OD) δ1.28 (t, 3H, J=7.6 Hz, CH₃), 2.72 (q, 2H, J=7.6 Hz, CH₂), 7.21 (br s, 2H, ArH), 7.35 (d, 2H, J=8.2 Hz, ArH), 7.53-7.55 (m, 4H, ArH), 7.91 (d, 2H, J=8.2 Hz, ArH), 8.44 (br s, 2H, ArH); ¹³C NMR (150 MHz, CD₃OD) δ16.2, 29.9, 117.0 (²J_{CF}=22.4 Hz), 123.3, 127.3, 128.5 (⁴J_{CF}=4.5 Hz), 129.4, 129.6, 132.3 (³J_{CF}=7.9 Hz), 135.1, 143.6, 147.3, 149.7, 150.3, 164.5 (¹J_{CF}=246.9 Hz); HRMS (ESI): calcd for C₂₂H₁₉FN₃ 344.1558, found 344.1572 [MH]⁺.

2-(4-iso-Butylphenyl)-4-(4-fluorophenyl)-5-(4-pyridyl)imidazole (12). This product was synthesized by reacting 4 with 4-iso-butylbenzaldehyde according to Method A. Purification by flash chromatography (silica, 100% CH₂C₁₂ to 96:4 CH₂Cl₂/MeOH) gave the product as a yellow crystalline solid (49%). ¹H NMR (600 MHz, CD₃OD) δ0.94 (d, 6H, J=6.8 Hz, CH₂CH(CH₃)₂), 1.92 (nonet, 1H, J=6.8 Hz, CH₂CH(CH₃)₂), 2.56 (d, 2H, J=6.8 Hz, CH₂CH(CH₃)₂), 7.21 (t, 2H, J=7.8 Hz, ArH), 7.30 (d, 2H, J=8.2 Hz, ArH), 7.53-7.55 (m, 4H, ArH), 7.91 (d, 2H, J=8.2 Hz, ArH), 8.43 (br s, 2H, ArH); ¹³C NMR (150 MHz, CD₃OD) δ22.9, 31.6, 46.3, 117.0 (d, ²J_{CF}=22.4Hz), 123.3, 127.1, 128.6, 129.3, 129.6, 130.4, 130.9, 132.3 (d, ³J_{CF}=7.9 Hz), 143.6, 144.7, 149.7, 150.2, 164.4 (d, ¹J_{CF}=248.0 Hz); HRMS (ESI): calcd for C₂₄H₂₃FN3 372.1871, found 372.1860 [MH]⁺.

1-Methoxymethyl-2-(4-iso-butylphenyl)-4,5-dibromoimidazole (6). Compound 5 (3.66 g, 10.2 mmol) and 4-iso-butylphenylboronic acid (1.89 g, 10.6 mmol) were dissolved in toluene/MeOH (5:1, 105.0 mL), and an aqueous solution of K₂CO₃ (2.0 M, 11.5 mL) was added. The mixture was degassed with argon for 20 minutes while stirring, followed by the addition of Pd(PPh₃)₄ (1.23 g, 1.1 mmol). The mixture was stirred at reflux for 18 h. It was cooled to room temperature and diluted with water (40 mL) and EtOAc (20 mL). The layers were separated, and the aqueous layer was extracted with EtOAc (3×20 mL). The combined organic layer was washed with brine (20 mL) and dried (Na2SO₄). The solvent was removed in vacuo, and the crude product was purified by flash chromatography (silica, 100% hexanes to 9:1 hexanes/EtOAc) to give a pale-yellow viscous oil (3.83 g, 86%). ¹H NMR (600 MHz, CDCl₃) δ0.91 (d, 6H, J=6.8 Hz, CH₂CH(CH₃)₂)), 1.89 (nonet, 1H, J=6.8 Hz, CH₂CH(CH₃)₂), 2.52 (d, 2H, J=6.8 Hz, CH₂CH(CH₃)₂), 3.44 (s, 3H, OCH₃), 5.27 (s, 2H, NCH₂O), 7.23 (d, 2H, J=8.2 Hz, ArH), 7.65 (d, 2H, J=8.2 Hz, ArH); ¹³C NMR (150 MHz, CDCl₃) δ22.5, 30.4, 45.4, 56.8, 76.4, 105.1, 118.0, 126.8, 128.8, 129.7, 144.1, 150; HRMS (ESI): calcd for C₁₅H₁₉Br₂N₂O 400.9859, found 400.9895 [MH]⁺.

1-Methoxymethyl-2-(4-iso-butylphenyl)-4-tributylstannyl-5-bromoimidazole (7). n-Butyl lithium (1.6 M in hexanes, 6.1 mL, 9.8 mmol) was added dropwise to a solution of 6 (3.76 g, 9.4 mmol) in THF (46.0 mL) at −78° C., and the mixture was stirred at this temperature for 15 min. Tri-n-butyltin chloride (2.8 mL, 10.3 mmol) was then added dropwise, and the reaction mixture was stirred at −78° C. for 30 min, before being poured into saturated NaHCO₃ (40 mL). The aqueous layer was extracted with EtOAc (3×20 mL) and the combined organic layer was dried over anhydrous Na₂SO₄. The suspension was filtered and the solvent in the filtrate was evaporated to dryness in vacuo. The residue was purified by column chromatography (silica, 100% hexanes to 95:5 hexanes/EtOAc) to give the product as a pale-orange viscous oil (3.92 g, 68%). ¹H NMR (600 MHz, CDCl₃) δ0.90-0.92 (m, 15H, 5×CH₃), 1.20-1.23 (m, 6H, 3×CH₂), 1.36 (sextet, 6H, J=7.3 Hz, 3×CH₂), 1.54-1.59 (m, 6H, 3×CH₂), 1.89 (nonet, 1H, J=6.8 Hz, CH₂CH(CH₃)₂), 2.51 (d, 2H, J=6.8 Hz, CH₂CH(CH₃)₂), 3.09 (s, 3H, OCH₃), 5.14 (s, 2H, NCH₂O), 7.21 (d, 2H, J=8.2 Hz, ArH), 7.47 (d, 2H, J=8.2 Hz, ArH); ¹³C NMR (150 MHz, CDCl₃) δ11.0, 13.9, 22.5, 27.5, 29.1, 30.4, 45.4, 53.3, 77.6, 120.3, 127.3, 129.2, 129.5, 130.5, 143.2, 152.6.; HRMS (ESI): calcd for C₂₇H₄₆BrN₂OSn 613.1804, found 613.1839 [MH]⁺.

1-Methoxymethyl-2-(4-iso-butylphenyl)-4-bromo-5-(4-pyridyl)imidazole (8). Stannane 7 (1.61 g, 2.6 mmol), 4-iodopyridine (0.60 g, 2.9 mmol), and Pd(PPh₃)₄ (0.61 g, 0.53 mmol) were dissolved in DMF (26.5 mL), and argon was bubbled through the mixture for 20 min. It was then heated at 110° C. for 45 h, at which point it was cooled to room temperature and was poured into water (30 mL) and extracted with EtOAc (3×15 mL). The combined organic layer was washed with brine (15 mL) and dried over anhydrous Na₂SO₄. The suspension was filtered and the filtrate was evaporated to dryness in vacuo. Purification by column chromatography (100% hexanes to 1:1 hexanes/EtOAc) gave the title compound as a sticky solid (0.86 g, 82%). ¹H NMR (600 MHz, CDCl₃) δ0.92 (d, 6H, J=6.8 Hz, CH₂CH(CH₃)₂), 1.91 (nonet, 1H, J=6.8 Hz, CH₂CH(CH₃)₂), 2.54 (d, 2H, J=6.8 Hz, CH₂CH(CH₃)₂), 3.32 (s, 3H, OCH₃), 4.99 (s, 2H, NCH₂O), 7.27 (d, 2H, J=8.2 Hz, ArH), 7.62 (d, 2H, J=5.4 Hz, ArH), 7.71 (d, 2H, J=8.2 Hz, ArH), 7.75 (d, 2H, J=5.4 Hz, ArH); ¹³C NMR (150 MHz, CDCl₃) δ22.5, 30.4, 45.4, 55.4, 75.8, 117.1, 124.0, 126.4, 128.9, 129.8, 132.3, 136.5, 144.2, 150.4, 150.9; HRMS (ESI): calcd for C₂₃H₂₃BrN₃O 400.1019, found 400.1046 [MH]⁺.

1-Methoxymethyl-2-(4-iso-butylphenyl)-4-bromo-5-(4-fluorophenyl)imidazole (9). Stannane 7 (1.70 g, 2.8 mmol) and 4-fluoroiodobenzene (0.32 mL, 2.8 mmol) were dissolved in DMF (73.0 mL) and argon was bubbled through the mixture for 20 min. Tris(dibenzylidene-acetone)dipalladium(0)-chloroform adduct (Pd₂(dba)₃.CHCl₃, 0.45 g, 0.44 mmol), AsPh₃ (0.68 g, 2.2 mmol), and Cul (1.36 g, 7.1 mmol) were then added, and the mixture was stirred at room temperature for 2 h. It was then poured into water (200 mL) and the solution was extracted with EtOAc (3×75 mL). The combined organic layer was washed with water (2×50 mL) and brine (50 mL), then dried over anhydrous Na₂SO₄. After filtration and removal of the solvent from the filtrate in vacuo, the crude product was purified by column chromatography (silica, 100% hexanes to 9:1 hexanes/EtOAc) and recrystallization from EtOAc/hexanes to give off-white crystals. (0.87 g, 75%). ¹H NMR (600 MHz, CDCl₃) δ0.92 (d, 6H, J=6.8 Hz, CH₂CH(CH₃)₂), 1.91 (nonet, 1H, J=6.8 Hz, CH₂CH(CH₃)₂), 2.53 (d, 2H, J=6.8 Hz, CH₂CH(CH₃)₂), 3.25 (s, 3H, OCH₃), 4.97 (s, 2H, NCH₂O), 7.19 (t, 2H, J=8.7 Hz, ArH), 7.25 (d, 2H, J=8.4 Hz, ArH), 7.59 (dd, 2H, J=8.7, 5.3 Hz, ArH), 7.70 (d, 2H, J=8.4 Hz, ArH); ¹³C NMR (150 MHz, CDCl₃) δ22.5, 30.4, 45.4, 55.6, 75.6, 115.6, 116.0 (d, ²J_{CF}=22.4 Hz), 124.7 (d, ⁴J_{CF}=3.4 Hz), 126.9, 128.8, 129.7, 130.7, 132.4 (d, ³J_{CF}=7.9 Hz), 143.7, 149.5, 163.1 (d, ¹J_{CF}=249.1 Hz); HRMS (ESI): calcd for C₂₁H₂₃BrFN₂O 417.0972, found 417.0981 [MH]⁺.

### Example 2

### Phosphorylation of BlaR1 in Manifestation of the Methicillin-Resistance Phenotype in Staphylococcus aureus and its Reversal by Small Molecules

Experimental Procedures.

Bacterial strains. Staphylococcus aureus strain MRSA252 was obtained from the American Type Culture Collection (ATCC, Manassas, Va., U.S.A.); S. aureus strains NRS70, NRS123, and NRS128 were acquired from the Network on Antimicrobial Resistance in Staphylococcus aureus (NARSA, Chantilly, Va., U.S.A.).

Minimal-inhibitory concentration (MIC) determination. Determination of MICs was done by the microdilution method cation-adjusted in Mueller-Hinton II Broth (CAMHB II, BBL) in accordance with the protocols of CLSI (Performance Standards for Antimicrobial Susceptibility Testing; Twenty-Second Informational Supplement. CLSI document M100-S22. Clinical and Laboratory Standards Institute Wayne, Pa.). A final bacterial inoculum of 5×10⁵ CFU/mL was used, and the results were recorded after incubation for 16-20 h at 37° C.

Detection of BlaR1 phosphorylation in presence of antibiotic. As we had disclosed in a recent publication on fragmentation of BlaR1 during the course of induction, we detect a cleavage at position S283-F284 of BlaR1 (Llarrull et al., J. Biol. Chem. 2011, 286, 38148-38158). This proteolytic cleavage cuts the BlaR1 protein into two fragments of roughly equal sizes (approx. 30-31 kDa). Zhang et al. identified another fragmentation site nearby, namely R293-R294 (Science 2001, 291, 1962-1965). In order to process these protein samples for identification of the phosphorylation sites, Staphylococcus aureus NRS128 was grown in LB media to OD₆₂₅=0.7, then was allowed to grow an additional three hours at 37° C. in the absence or presence of 10 μg/mL CBAP, a good inducer of the bla system.

Extracts were prepared as previously described in buffer containing 50 mM Tris pH 7.5, 150 mM NaCl, 2 mM EDTA, 0.55% SDS, 2.5% Triton X-100, and Halt Protease and Phosphatase Inhibitor Cocktail (Thermo Scientific, Waltham, Mass., U.S.A.) and analyzed by Western blot using antibodies against phosphothreonine, phosphotyrosine, and phosphoserine. A ˜30 kDa protein band was detected by the phosphotyrosine and phosphoserine specific antibodies only in cell extracts of S. aureus cells grown with CBAP (FIG. 2). This band was not detected by the phosphothreonine antibody.

Initially, we had an abundance of Protein A and other immunoglobulin-binding proteins detected in the western blot, which ran in the range of 40-60 kDa and precluded visualization of the full-length BlaR1. To overcome this, we subsequently cleared all S. aureus extracts of Protein A and other immunoglobulin-binding proteins by incubation with IgG Sepharose (GE Healthcare, Little Chalfont, UK) for 1-2 h at room temperature with gentle agitation. After a brief centrifugation, the total protein in the supernatant was quantified by a BCA assay and 20 μg was loaded onto an 11% SDS-PAGE gel. Following electrophoresis, samples were transferred to a nitrocellulose membrane in a 10 mM CAPS (pH 11) buffer containing 10% methanol. Membranes were blocked in 3% BSA for phosphoserine blots or Blotto (3% BSA, 3% milk in TBS) for phosphotyrosine blots. HRP-conjugated primary antibody was applied in either 1% BSA/TBST (phosphoserine, Abcam, Cambridge, UK) or 1.1% milk/TBST (phosphotyrosine, 4G10 Platinum, EMD Millipore, Billerica, Mass., U.S.A.). Phosphoserine blots were developed with Pierce ECL Substrate (Thermo Scientific) and phosphotyrosine blots were developed with SuperSignal West Dura Extended Duration Substrate (Thermo Scientific), then exposed to X-ray film for an appropriate amount of time (30-600 s).

Monitoring β-lactamase expression by nitrocefin assay. The media from CBAP-induced cultures grown in the absence or presence of 7 μg/mL compounds 10, 11, or 12 was separated from the cells by centrifugation at 3200 g, at 4° C. for 30 min. The absorbance of hydrolyzed chromogenic nitrocefin was monitored at 500 nm at room temperature for 5 min by adding 100 μM nitrocefin to 1 mL culture media. The initial rates of the reactions were determined by linear regression and the activity was normalized to the activity in the absence of inhibitor to give % activity.

Identification of phosphorylated domain of BlaR1. As the two fragments of proteolyzed BlaR1 are roughly the same size, it is difficult to determine the site of phosphorylation without separation. To accomplish this, we immunoprecipitated whole-cell extracts of NRS128 grown in the absence and presence of CBAP using an antibody raised against the sensor domain of BlaR1 (BlaR^{s}) immobilized on Protein A-agarose. BlaR^{s}-Agarose resin for immunoprecipitation of S. aureus extracts was prepared as previously described (Llarrull et al., J. Biol. Chem. 2011, 286, 38148-38158) by crosslinking BlaR^{s} antibody to Protein A Agarose beads (Thermo Scientific) with dimethylpimelimidate (DMP) in sodium phosphate buffer (pH 7.4) with 150 mM NaCl. Crosslinked resin was stored in PBS at 4° C.

Whole-cell extracts were first cleared of Protein A and other immunoglobulin-binding proteins (as described above), then were incubated with BlaR^{s}-Agarose resin overnight at 4° C. with end-over-end rotation. Unbound protein was removed after centrifugation. Bound proteins (containing BlaR^{s}, including full-length BlaR1) were eluted from the resin using Laemmli sample buffer. Unbound and bound fractions were loaded onto an 11% SDS-PAGE gel and subjected to western blot analysis with phosphotyrosine, phosphoserine, and BlaR^{s} antibodies (FIG. 3).

Probing with the BlaR^{s} antibody revealed the C-terminal BlaR1 fragment that contains the sensor domain only in the bound fraction (band at ˜30 kDa; arrow), while it revealed the full-length BlaR1 both in the bound and unbound fractions (band at ˜60 kDa; arrows). The presence of the full-length BlaR1 in the unbound fraction is not unexpected since its interaction with lipids (it has four transmembrane helixes) makes its affinity-purification less efficient. Probing with the phosphotyrosine antibody also revealed a band at ˜60 kDa both in the unbound and bound fractions. The correlation of the intensity of the full-length BlaR1 bands in the unbound and bound protein fractions of the membranes probed with the BlaR^{s} antibody, with the intensity of the bands at ˜60 kDa in the unbound and bound protein fractions of the membranes probed with the phosphotyrosine antibody indicates that the phosphorylated protein is BlaR1. Probing with the phosphotyrosine antibody revealed a ˜30 kDa fragment only in the unbound fraction. This indicated that the site of tyrosine phosphorylation is located in the N-terminal fragment of BlaR1 (residues 1-283), which contains the cytoplasmic protease domain.

This observation makes good sense, as ATP, the source of phosphate, is found only in the cytoplasm. A repeat of these experiments with anti-phosphoserine antibody indicates that the N-terminal half is also phosphorylated at a serine (FIG. 3c). Incidentally, the protease domain contains eleven serine and ten tyrosine residues, any of which could be the sites of phosphorylation.

Cloning, expression, and purification of S. aureus Stk1 protein kinase. The stk1 gene for Stk1 protein kinase (SA1063 in S. aureus NRS70) is conserved in all known genomic sequences for S. aureus (Beltramini et al., Infect. Immun. 77, 1406-1416 (2009); Débarbouillé et al., J. Bacteriol. 191, 4070-4081 (2009); Didier et al., FEMS Microbiol. Lett. 306, 30-36 (2010)). We PCR-amplified the DNA fragment corresponding to the entire coding sequences of stk1 from S. aureus strain NRS70 chromosomal DNA with primers STK1fw (5′-CCCCCCCATATG ATAGGTAAAATAATAAATGAACGATAT-3′) and STK1rev (5′-CCCCCCCTCGAG TTAAATATCATCATAGCTGACTTCTTTTTC-3′). The amplified DNA fragments were then digested with NdeI and XhoI restriction enzymes and cloned into pET28a. After verification of the inserts by DNA sequencing on both strands, the resulting plasmid (termed pETstk1) was introduced into E. coli strain BL21(DE3) for protein expression.

For purification of Stk1, 5 mL of an overnight culture of BL21(DE3) harboring pETstk1 was inoculated into 500 mL fresh LB medium and grown at 37° C. until the OD₆₀₀ reached 0.8. Isopropyl β-D-1-thiogalactopyranoside (IPTG) was then added to the culture to a final concentration of 0.5 mM and the culture was shaken at 15° C. overnight. The culture was then centrifuged at 5000 rpm at 4° C. for 15 min. The pellet was resuspended in 20 mL of lysis buffer (25 mM HEPES, 500 mM NaCl, 10 mM imidazole, pH 7.4). After sonication, the lysate was centrifuged at 18,000 g at 4° C. for 45 min. The resulting supernatant containing the His-tagged Stk1 was loaded onto a 5-mL Hitrap Chelating column (GE Healthcare), followed by elution with a linear gradient of imidazole (0-500 mM) in lysis buffer. The fractions containing Stk1 were pooled, concentrated and the buffer was exchanged to 25 mM HEPES, pH 7.4. The resulting sample was then subjected to Q anion-exchange chromatography and eluted with a linear gradient of NaCl (0-1 M). Protein purity was ascertained by sodium dodecyl sulfate polyacrylamide gel electrophoresis (SDS-PAGE) (FIG. 7).

In vitro phosphorylation assay. The Stk1 protein kinase was assayed for its autophosphorylation (on serine and threonine residues) and for phosphorylation of myelin basic protein (MBP) (a commercially available, nonspecific substrate of Ser/Thr protein kinases). The assay was used for assessment of inhibition of the protein by the synthetic kinase inhibitors 10-12. The reaction was carried out in 20 μL of volume containing 1 μg purified Stk1, 4 μg MBP, varying concentrations of compound 10-12, in 25 mM Tris, pH 7.4, 1 mM dithiothreitol and 10 mM MgCl₂, initiated by the addition of 4 μCi [γ-³²P]-ATP (20 μM). The assay mixture was incubated at room temperature for 20 min and it was stopped by the addition of 5×SDS-PAGE sample buffer. After boiling for 5 minutes, the mixtures were subjected to SDS-PAGE. The gel was then exposed to storage phosphor screen overnight and the screen was scanned with an Amersham Storm 840. Band intensities were quantified using GelQuant software. The band intensities in the presence of compounds 10-12 were divided by the intensities in the absence of inhibitors to obtain the relative band intensities. Relative band intensities of the Stk1 or MBP bands were plotted against the concentration of compounds 10-12 (μM) (FIG. 8) and GraphPad Prism 5 was used to calculate the IC₅₀ values by non-linear regression, using the equation Y=IC₅₀/[IC₅₀+X] as previously described (Dzhekieva et al., Biochemistry 51, 2804-2811 (2012)), with R² values ranging from 0.87 to 0.91.

### Example 3

### Compound MIC Data

The MIC values of more than 80 compounds were evaluated according to the methods described above. Each compound was evaluated at three or more different concentrations, typically: 0 (control), 2, and 20 μM. If the compound exhibited inherent antibacterial activity at 20 μM against the S. aureus strain, the compound was reevaluated at lower concentrations for the particular strain (e.g., 0, 0.1, and 1μM, or 0, 5, and 10 μM) to eliminate interference with the assay. Evaluation of compounds 1, A3, A4, A6, A10, A11, A12, A13, A14, A15, A16, A17, A18, A20, A21, A22, A34, A35, A37, A40, A42, A45, A53, A54, and A61 showed a greater than 2-fold decrease relative to the control.

### Compounds Numbering:

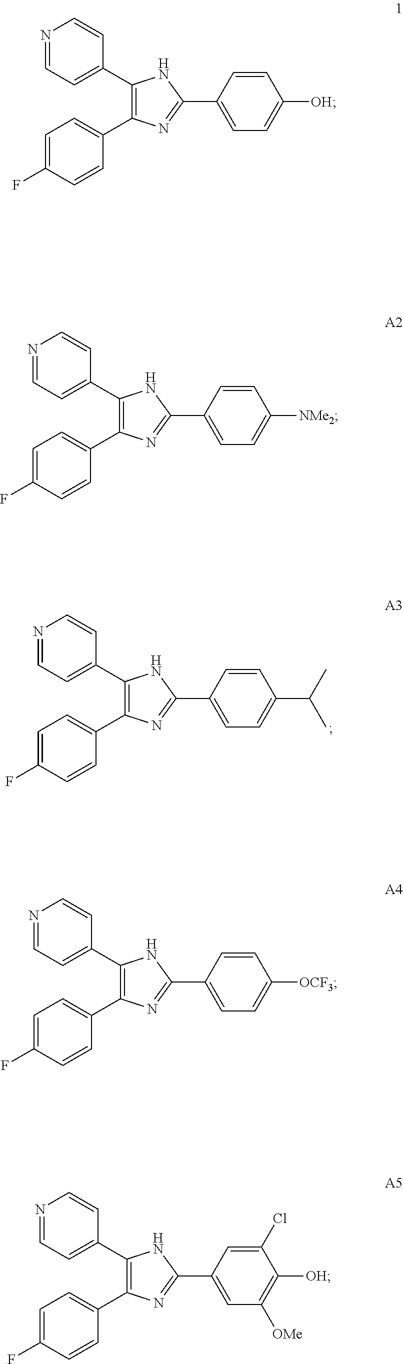 [SMILES: Oc1ccc(-c2nc(-c3ccc(F)cc3)c(-c3ccncc3)[nH]2)cc1 ; CN(C)c1ccc(-c2nc(-c3ccc(F)cc3)c(-c3ccncc3)[nH]2)cc1 ; CC(C)c1ccc(-c2nc(-c3ccc(F)cc3)c(-c3ccncc3)[nH]2)cc1 ; Fc1ccc(-c2nc(-c3ccc(OC(F)(F)F)cc3)[nH]c2-c2ccncc2)cc1 ; COc1cc(-c2nc(-c3ccc(F)cc3)c(-c3ccncc3)[nH]2)cc(Cl)c1O]

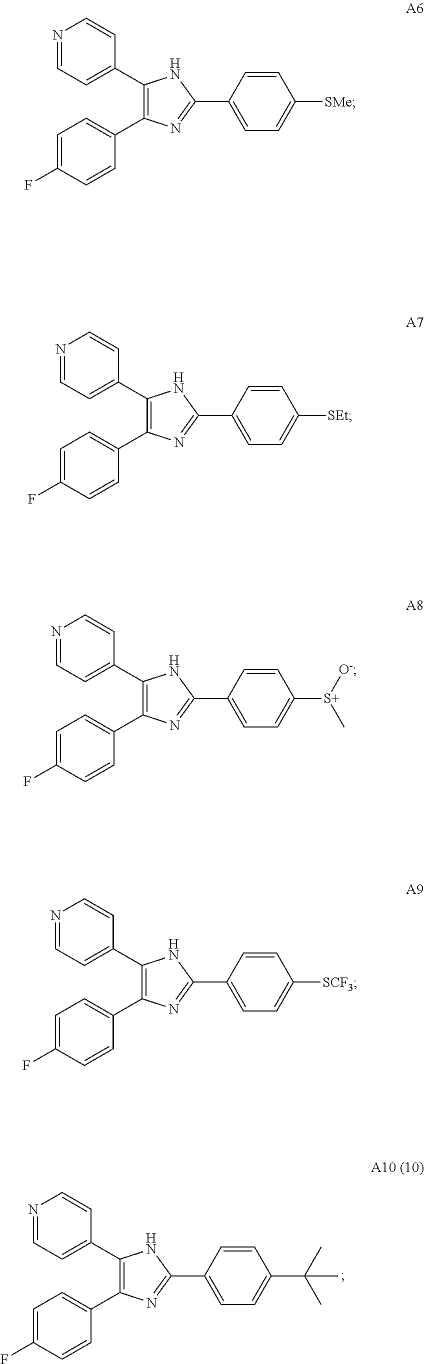

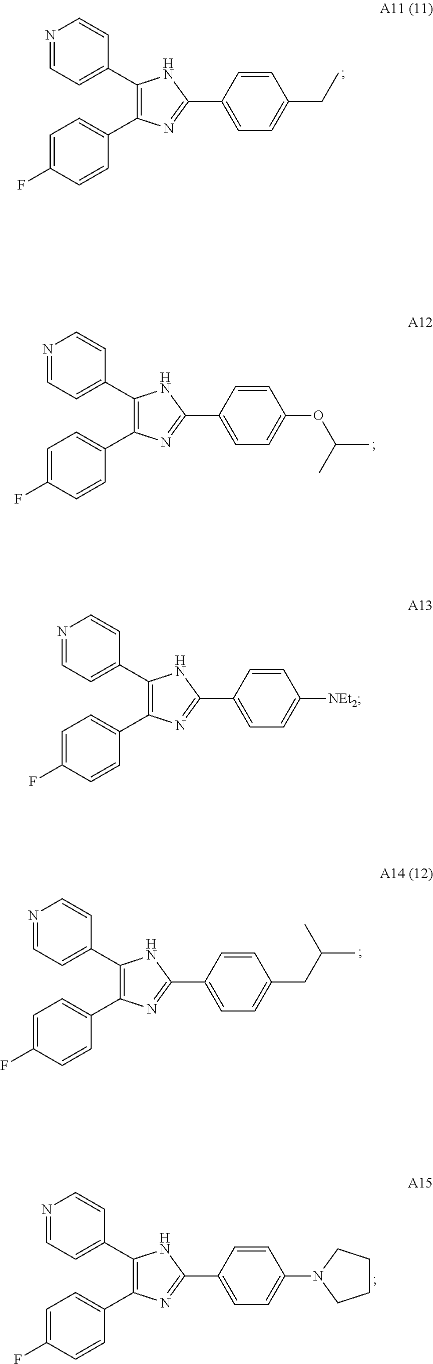

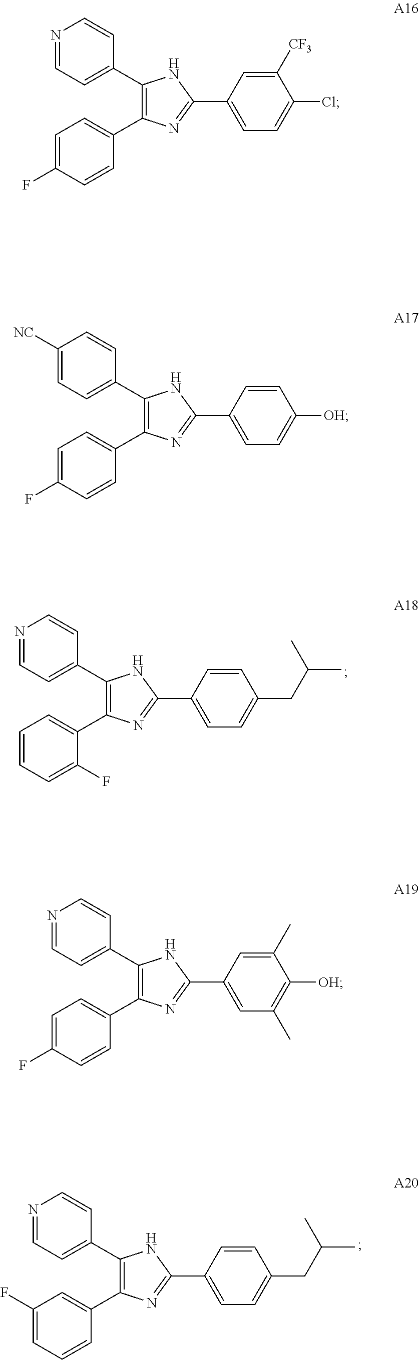 [SMILES: Fc1ccc(-c2nc(-c3ccc(Cl)c(C(F)(F)F)c3)[nH]c2-c2ccncc2)cc1 ; N#Cc1ccc(-c2[nH]c(-c3ccc(O)cc3)nc2-c2ccc(F)cc2)cc1 ; CC(C)Cc1ccc(-c2nc(-c3ccccc3F)c(-c3ccncc3)[nH]2)cc1 ; Cc1cc(-c2nc(-c3ccc(F)cc3)c(-c3ccncc3)[nH]2)cc(C)c1O ; CC(C)Cc1ccc(-c2nc(-c3cccc(F)c3)c(-c3ccncc3)[nH]2)cc1]

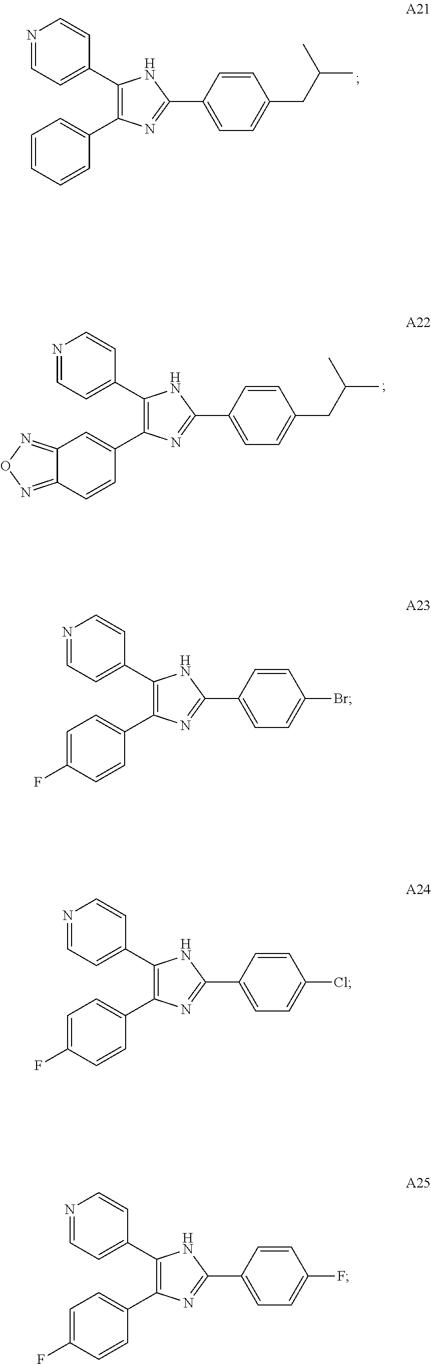 [SMILES: CC(C)Cc1ccc(-c2nc(-c3ccccc3)c(-c3ccncc3)[nH]2)cc1 ; CC(C)Cc1ccc(-c2nc(-c3ccc4nonc4c3)c(-c3ccncc3)[nH]2)cc1 ; Fc1ccc(-c2nc(-c3ccc(Br)cc3)[nH]c2-c2ccncc2)cc1 ; Fc1ccc(-c2nc(-c3ccc(Cl)cc3)[nH]c2-c2ccncc2)cc1 ; Fc1ccc(-c2nc(-c3ccc(F)cc3)c(-c3ccncc3)[nH]2)cc1]

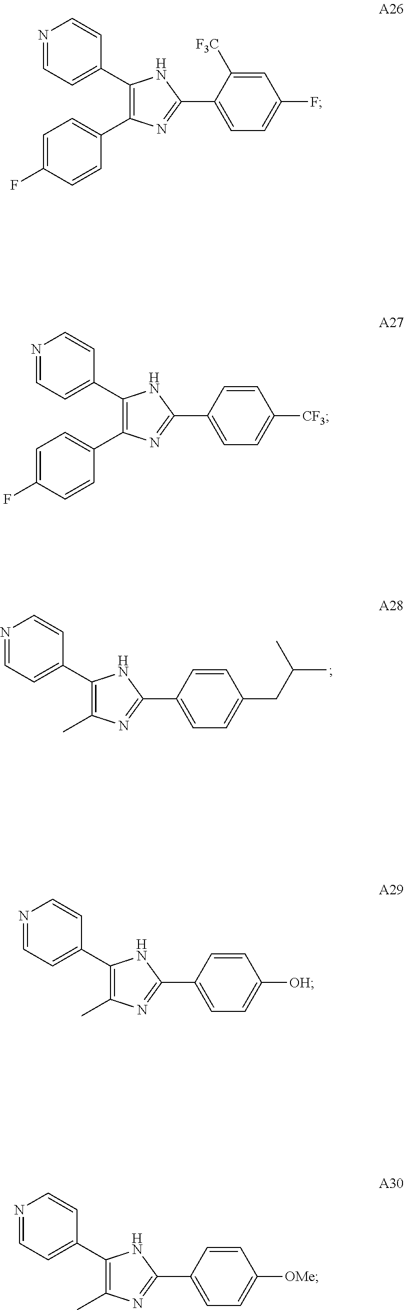 [SMILES: Fc1ccc(-c2nc(-c3ccc(F)cc3C(F)(F)F)[nH]c2-c2ccncc2)cc1 ; Fc1ccc(-c2nc(-c3ccc(C(F)(F)F)cc3)[nH]c2-c2ccncc2)cc1 ; Cc1nc(-c2ccc(CC(C)C)cc2)[nH]c1-c1ccncc1 ; Cc1nc(-c2ccc(O)cc2)[nH]c1-c1ccncc1 ; COc1ccc(-c2nc(C)c(-c3ccncc3)[nH]2)cc1]

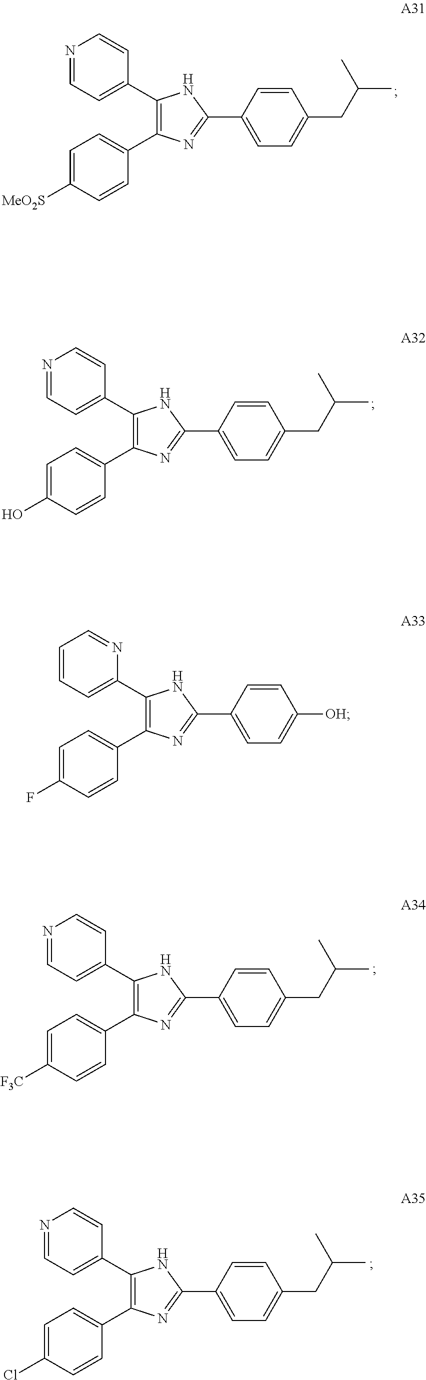 [SMILES: CC(C)Cc1ccc(-c2nc(-c3ccc(S(C)(=O)=O)cc3)c(-c3ccncc3)[nH]2)cc1 ; CC(C)Cc1ccc(-c2nc(-c3ccc(O)cc3)c(-c3ccncc3)[nH]2)cc1 ; Oc1ccc(-c2nc(-c3ccc(F)cc3)c(-c3ccccn3)[nH]2)cc1 ; CC(C)Cc1ccc(-c2nc(-c3ccc(C(F)(F)F)cc3)c(-c3ccncc3)[nH]2)cc1 ; CC(C)Cc1ccc(-c2nc(-c3ccc(Cl)cc3)c(-c3ccncc3)[nH]2)cc1]

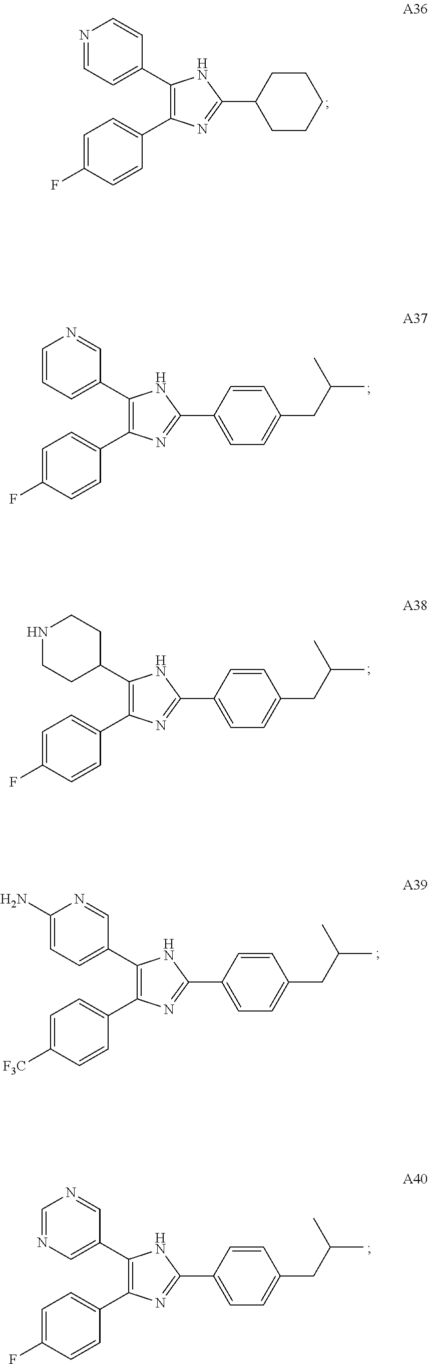 [SMILES: Fc1ccc(-c2nc(C3CCCCC3)[nH]c2-c2ccncc2)cc1 ; CC(C)Cc1ccc(-c2nc(-c3ccc(F)cc3)c(-c3cccnc3)[nH]2)cc1 ; CC(C)Cc1ccc(-c2nc(-c3ccc(F)cc3)c(C3CCNCC3)[nH]2)cc1 ; CC(C)Cc1ccc(-c2nc(-c3ccc(C(F)(F)F)cc3)c(-c3ccc(N)nc3)[nH]2)cc1 ; CC(C)Cc1ccc(-c2nc(-c3ccc(F)cc3)c(-c3cncnc3)[nH]2)cc1]

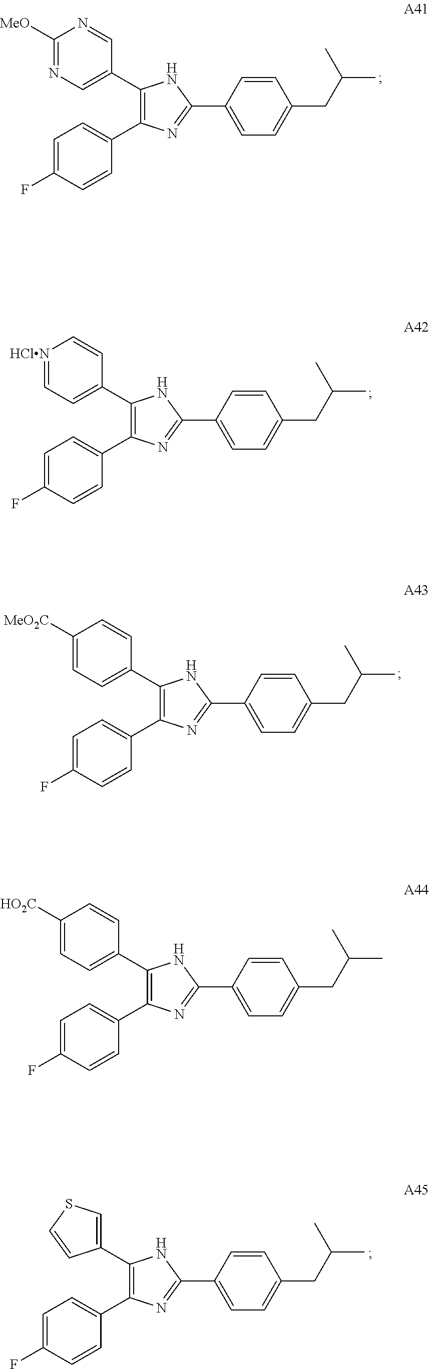

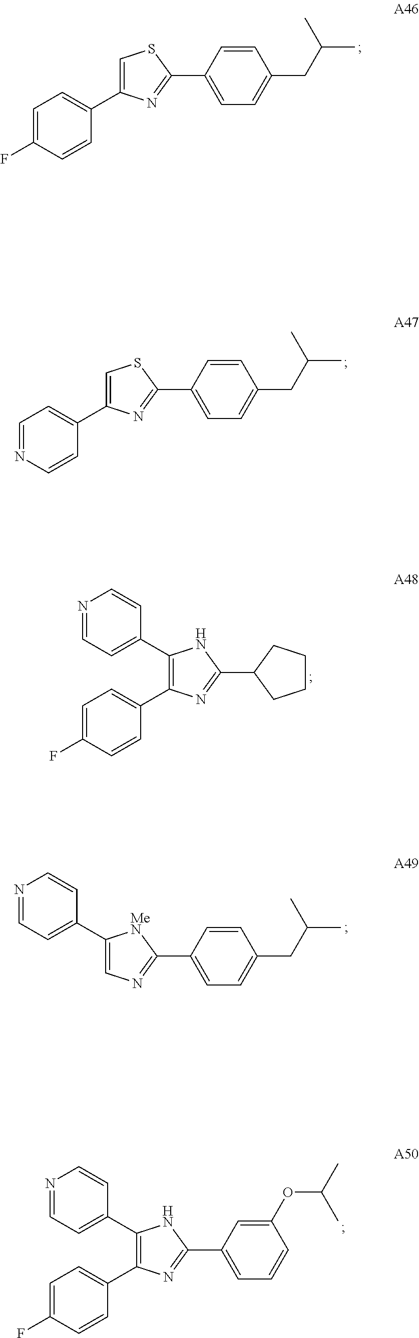 [SMILES: CC(C)Cc1ccc(-c2nc(-c3ccc(F)cc3)cs2)cc1 ; CC(C)Cc1ccc(-c2nc(-c3ccncc3)cs2)cc1 ; Fc1ccc(-c2nc(C3CCCC3)[nH]c2-c2ccncc2)cc1 ; CC(C)Cc1ccc(-c2ncc(-c3ccncc3)n2C)cc1 ; CC(C)Oc1cccc(-c2nc(-c3ccc(F)cc3)c(-c3ccncc3)[nH]2)c1]

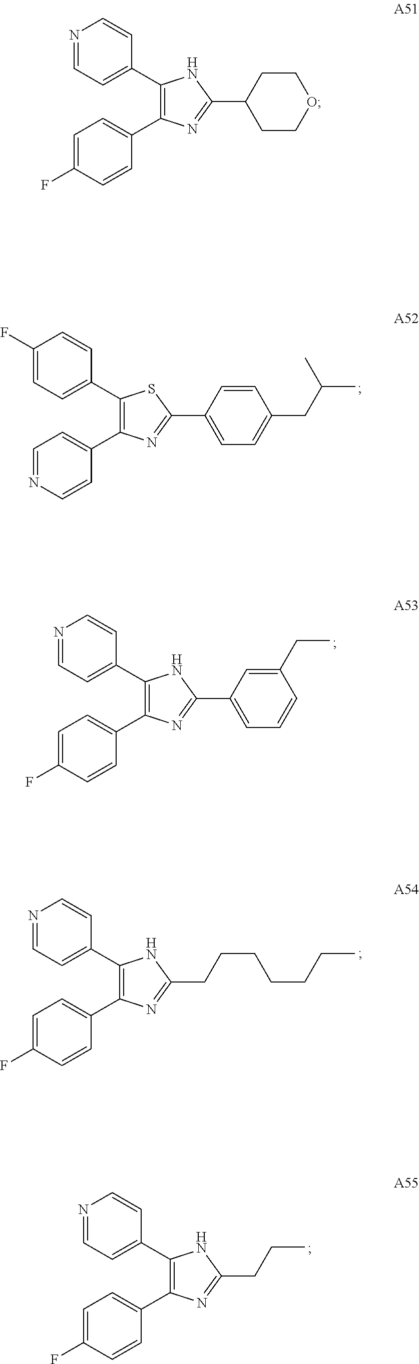 [SMILES: Fc1ccc(-c2nc(C3CCOCC3)[nH]c2-c2ccncc2)cc1 ; CC(C)Cc1ccc(-c2nc(-c3ccncc3)c(-c3ccc(F)cc3)s2)cc1 ; CCc1cccc(-c2nc(-c3ccc(F)cc3)c(-c3ccncc3)[nH]2)c1 ; CCCCCCCc1nc(-c2ccc(F)cc2)c(-c2ccncc2)[nH]1 ; CCCc1nc(-c2ccc(F)cc2)c(-c2ccncc2)[nH]1]

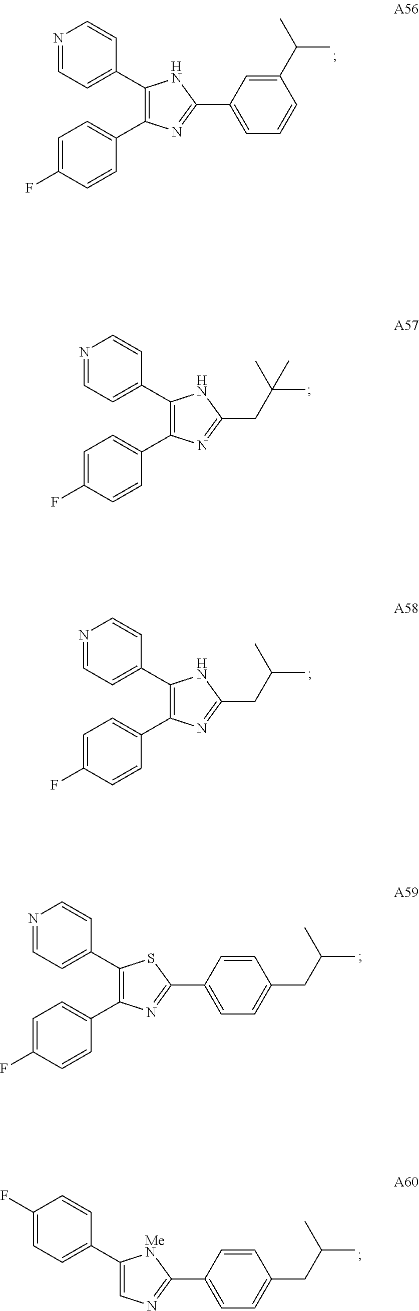 [SMILES: CC(C)c1cccc(-c2nc(-c3ccc(F)cc3)c(-c3ccncc3)[nH]2)c1 ; CC(C)(C)Cc1nc(-c2ccc(F)cc2)c(-c2ccncc2)[nH]1 ; CC(C)Cc1nc(-c2ccc(F)cc2)c(-c2ccncc2)[nH]1 ; CC(C)Cc1ccc(-c2nc(-c3ccc(F)cc3)c(-c3ccncc3)s2)cc1 ; CC(C)Cc1ccc(-c2ncc(-c3ccc(F)cc3)n2C)cc1]

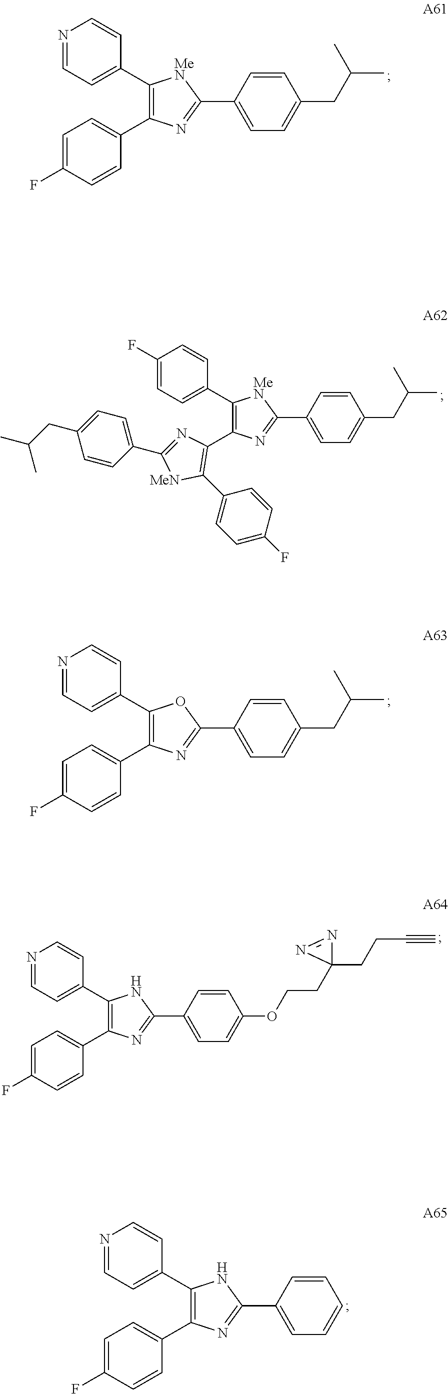

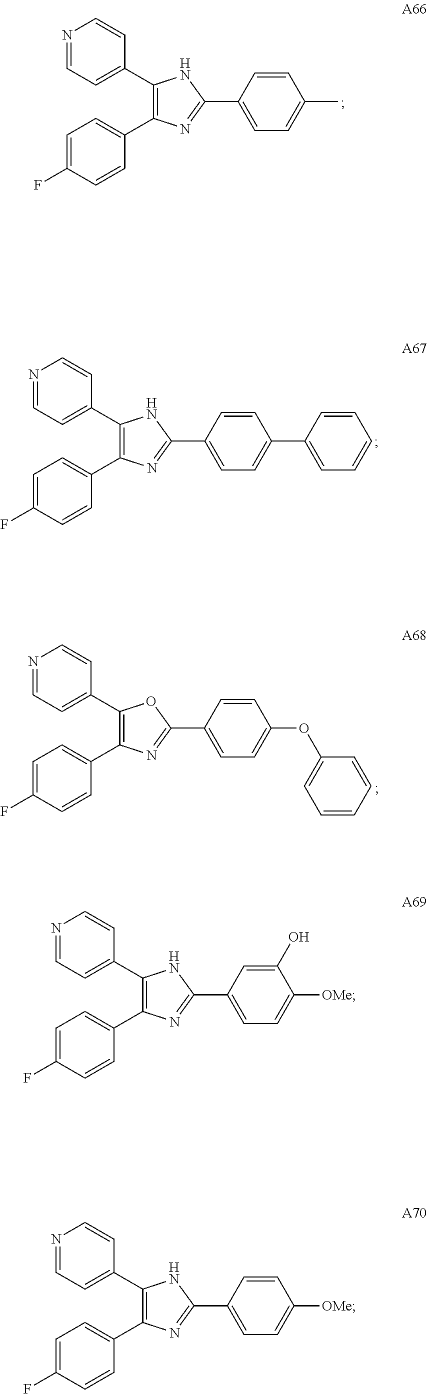 [SMILES: Cc1ccc(-c2nc(-c3ccc(F)cc3)c(-c3ccncc3)[nH]2)cc1 ; Fc1ccc(-c2nc(-c3ccc(-c4ccccc4)cc3)[nH]c2-c2ccncc2)cc1 ; Fc1ccc(-c2nc(-c3ccc(Oc4ccccc4)cc3)oc2-c2ccncc2)cc1 ; COc1ccc(-c2nc(-c3ccc(F)cc3)c(-c3ccncc3)[nH]2)cc1O ; COc1ccc(-c2nc(-c3ccc(F)cc3)c(-c3ccncc3)[nH]2)cc1]

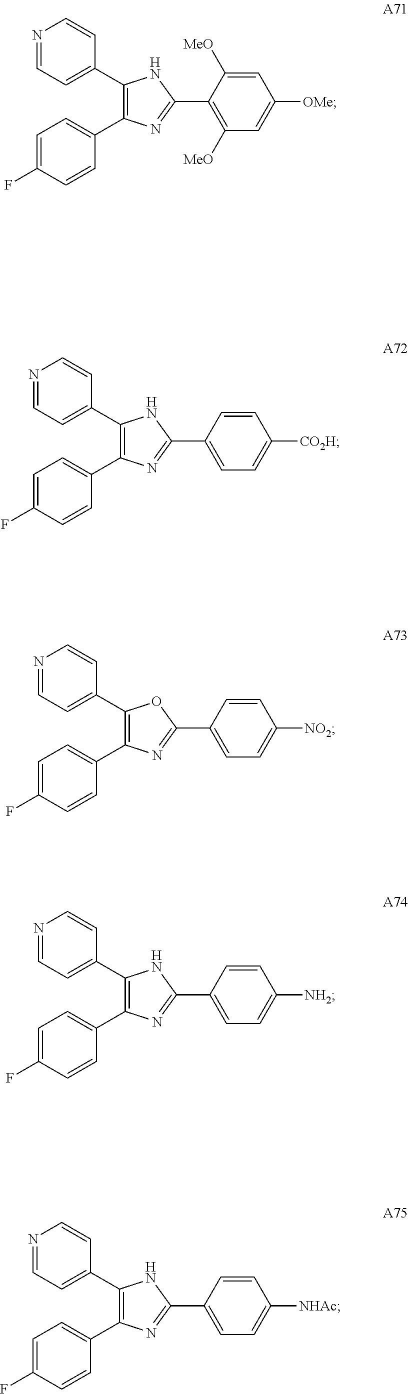 [SMILES: COc1cc(OC)c(-c2nc(-c3ccc(F)cc3)c(-c3ccncc3)[nH]2)c(OC)c1 ; O=C(O)c1ccc(-c2nc(-c3ccc(F)cc3)c(-c3ccncc3)[nH]2)cc1 ; O=[N+]([O-])c1ccc(-c2nc(-c3ccc(F)cc3)c(-c3ccncc3)o2)cc1 ; Nc1ccc(-c2nc(-c3ccc(F)cc3)c(-c3ccncc3)[nH]2)cc1 ; CC(=O)Nc1ccc(-c2nc(-c3ccc(F)cc3)c(-c3ccncc3)[nH]2)cc1]

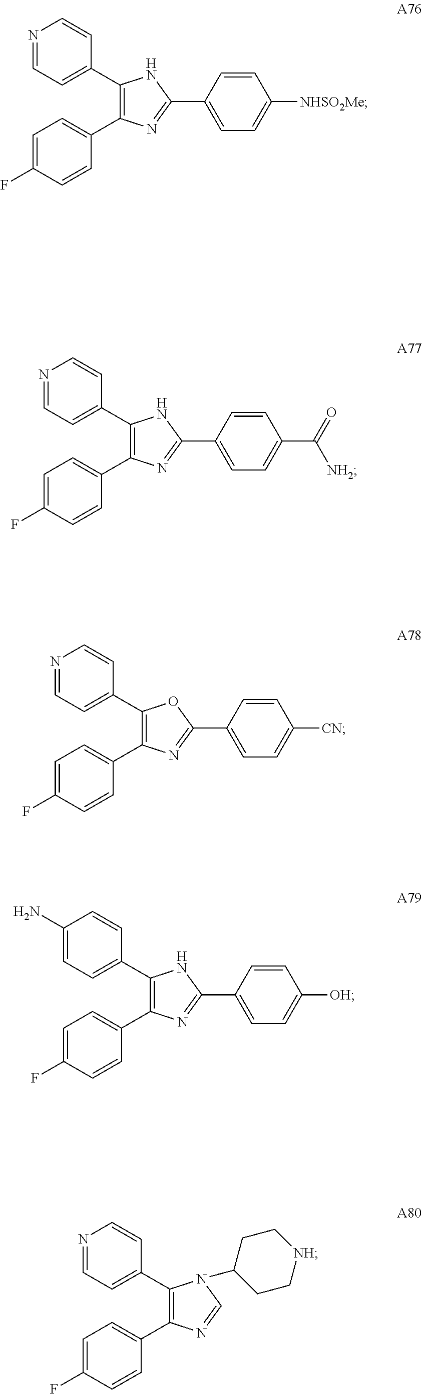

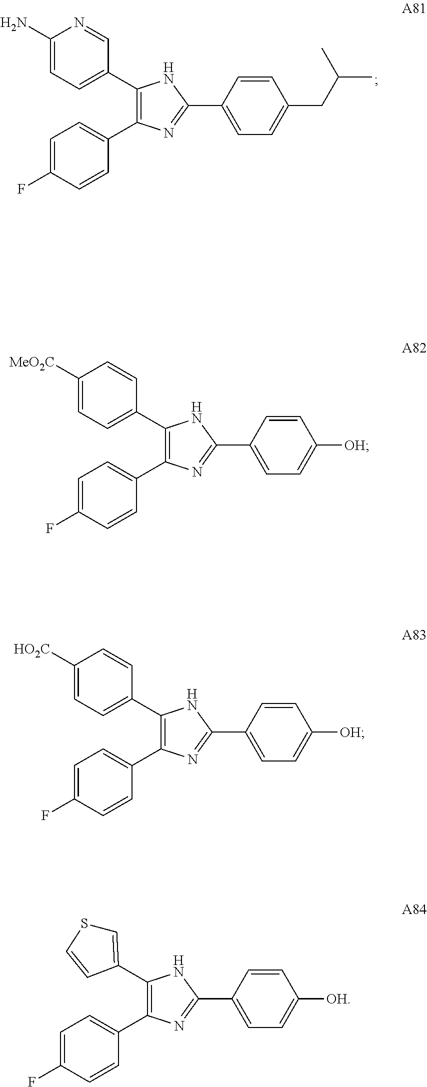 [SMILES: CC(C)Cc1ccc(-c2nc(-c3ccc(F)cc3)c(-c3ccc(N)nc3)[nH]2)cc1 ; COC(=O)c1ccc(-c2[nH]c(-c3ccc(O)cc3)nc2-c2ccc(F)cc2)cc1 ; O=C(O)c1ccc(-c2[nH]c(-c3ccc(O)cc3)nc2-c2ccc(F)cc2)cc1 ; Oc1ccc(-c2nc(-c3ccc(F)cc3)c(-c3ccsc3)[nH]2)cc1]

### Example 4

### Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of Formula I: 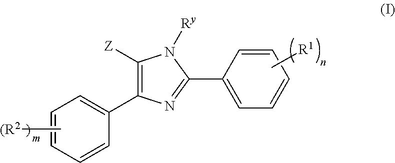
wherein
each R¹ is independently hydroxy, halo, (C₁-C₁₂)alkyl, (C₁-C₁₂)alkoxy, —CF₃, —OCF₃, —SH, —SMe, —N((C₂-C₈)alkyl)₂, or N-pyrrolidine;
each R² is independently H, hydroxy, halo, (C₁-C₁₂)alkyl, (C₁-C₁₂)alkoxy, —CF₃, or —OCF₃, or two R² groups form an oxadiazole;
R^{y} is H or (C₁-C₅)alkyl;
each n and m is independently 1, 2, 3, 4, or 5; and
Z is pyridyl, pyrimidinyl, thiophenyl, phenyl, or cyanophenyl;
provided that when Z is 4-pyridyl or R² is F in the para position, R¹ is not hydroxy, ethyl, isopropyl, tert-butyl, or —SMe in the para position;
or a pharmaceutically acceptable salt or solvate thereof.

2. The compound of claim 1 wherein Z is 4-pyridyl, 5-pyrimidinyl, 2-thiophenyl, 3-thiophenyl, phenyl, or 4-cyanophenyl.

3. The compound of claim 2 wherein the compound is a compound of Formula II: 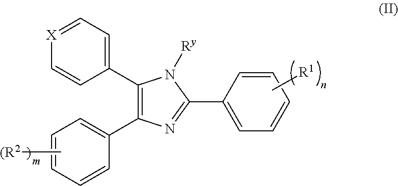
wherein
each R¹ is independently hydroxy, halo, (C₁-C₁₂)alkyl, (C₁-C₁₂)alkoxy, —CF₃, —OCF₃, —SH, —SMe, —N((C₂-C₈)alkyl)₂, or N-pyrrolidine;
each R² is independently H, hydroxy, halo, (C₁-C₁₂)alkyl, (C₁-C₁₂)alkoxy, —CF₃, or —OCF₃, or two R² groups form an oxadiazole;
R^{y} is H or (C₁-C₅)alkyl;
each n and m is independently 1, 2, 3, 4, or 5; and
X is N, CH, or C—CN;
or a pharmaceutically acceptable salt or solvate thereof.

4. The compound of claim 3 wherein X is N.

5. The compound of claim 3 wherein R¹ is hydroxy.

6. The compound of claim 3 wherein R¹ is (C₂-C₄)alkyl.

7. The compound of claim 3 wherein R¹ is ethyl, propyl, sec-propyl, iso-propyl, sec-butyl, or tert-butyl.

8. The compound of claim 3 wherein R² is halo.

9. The compound of claim 8 wherein R² is fluoro.

10. The compound of claim 3 wherein n is 1 and m is 1.

11. The compound of claim 3 wherein the compound is a compound of Formula III: 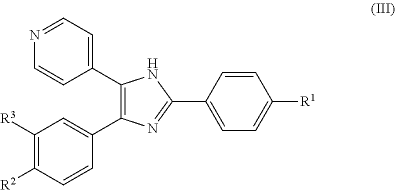
wherein
R¹ is hydroxy, halo, (C₁-C₁₂)alkyl, (C₁-C₁₂)alkoxy, —CF₃, —OCF₃, —SH, —SMe, —N((C₂-C₈)alkyl)₂, or N-pyrrolidine;
R² is H, hydroxy, halo, (C₁-C₁₂)alkyl, (C₁-C₁₂)alkoxy, —CF₃, or —OCF₃; and
R³ is H or F; or
R² and R³ together form an oxadiazole;
or a pharmaceutically acceptable salt or solvate thereof.

12. A composition comprising a compound of claim 1 in combination with a pharmaceutically acceptable diluent, excipient, or carrier.

13. A composition comprising a compound of claim 1 in combination with a β-lactam antibiotic.

14. The composition of claim 13 wherein the β-lactam antibiotic is oxacillin or ceftaroline.

15. A method to reverse the methicillin-resistant phenotype in BlaR1 comprising contacting methicillin-resistant Staphylococcus aureus (MRSA) with an effective amount of a compound of Formula I: 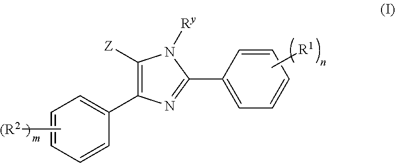
wherein
each R¹ is independently hydroxy, halo, (C₁-C₁₂)alkyl, (C₁-C₁₂)alkoxy, —CF₃, —OCF₃, —SH, —SMe, —N((C₂-C₈)alkyl)₂, or N-pyrrolidine;
each R² is independently H, hydroxy, halo, (C₁-C₁₂)alkyl, (C₁-C₁₂)alkoxy, —CF₃, or —OCF₃, or two R² groups form an oxadiazole;
R^{y} is H or (C₁-C₅)alkyl;
each n and m is independently 1, 2, 3, 4, or 5; and
Z is pyridyl, pyrimidinyl, thiophenyl, phenyl, or cyanophenyl;
or a pharmaceutically acceptable salt or solvate thereof;
thereby rendering MRSA susceptible to β-lactam antibiotics.

16. A method to inhibit or kill methicillin-resistant Staphylococcus aureus (MRSA) comprising contacting the MRSA with an amount of a compound of Formula I: 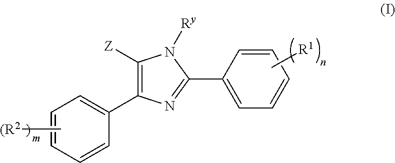
wherein
each R¹ is independently hydroxy, halo, (C₁-C₁₂)alkyl, (C₁-C₁₂)alkoxy, —CF₃, —OCF₃, —SH, —SMe, —N((C₂-C₈)alkyl)₂, or N-pyrrolidine;
each R² is independently H, hydroxy, halo, (C₁-C₁₂)alkyl, (C₁-C₁₂)alkoxy, —CF₃, or —OCF₃, or two R² groups form an oxadiazole;
R^{y} is H or (C₁-C₅)alkyl;
each n and m is independently 1, 2, 3, 4, or 5; and
Z is pyridyl, pyrimidinyl, thiophenyl, phenyl, or cyanophenyl;
or a pharmaceutically acceptable salt or solvate thereof; effective to reverse the methicillin-resistant phenotype in BlaR1, and contacting the MRSA with an effective antibacterial amount of a β-lactam antibiotic.

17. A method to lower the degree of phosphorylation of BlaR1 comprising contacting a bacteria having BlaR1 with an effective amount of a compound of claim 1
or a pharmaceutically acceptable salt or solvate thereof.

18. A method to attenuate or reduce the minimum inhibitory concentration (MIC) of a β-lactam antibiotic comprising contacting a bacterium with an effective amount of a compound of claim 1
or a pharmaceutically acceptable salt or solvate thereof; in combination with contacting the bacterium with a β-lactam antibiotic.

19. A method to treat a patient infected with a bacteria resistant to a β-lactam antibiotic comprising administering to the patient an effective amount of a compound of claim 1
or a pharmaceutically acceptable salt or solvate thereof; in combination with administering to the patient, concurrently or sequentially, an effective antibacterial amount of a β-lactam antibiotic.

20. The method of claim 19 wherein Z is 4-pyridyl, 5-pyrimidinyl, 2-thiophenyl, 3-thiophenyl, phenyl, or 4-cyanophenyl.

21.-31. (canceled)
